(19) United States
(12) Patent Application Publication
(10) Pub. No.: US 2024/0269124 A1
(43) Pub. Date: Aug. 15, 2024

(54) METHODS AND COMPOSITIONS FOR TREATING NEUROLOGICAL CONDITIONS

(71) Applicant: METANOIA BIO INC., Beverly Hills, CA (US)

(72) Inventors: Slavica TUDZAROVA-TRAJKOVSKA, Beverly Hills, CA (US); Oppel GREEFF, Beverly Hills, CA (US); Abraham VAN WYK, Beverly Hills, CA (US)

(21) Appl. No.: 18/561,247

(22) PCT Filed: May 16, 2022
(86) PCT No.: PCT/US2022/029406

§ 371 Date: Nov. 15, 2023

Related U.S. Application Data
(60) Provisional application No. 63/189,205, filed on May 16, 2021.

(51) Int. Cl. A61K 31/444; A61K 31/165; A61K 31/192; A61K 31/352; A61K 31/382; A61K 31/4045; A61K 31/4155; A61K 31/422; A61K 31/495; A61K 31/498; A61K 31/519; A61K 45/06

(52) U.S. Cl. CPC A61K 31/444; A61K 31/165; A61K 31/192; A61K 31/352; A61K 31/382; A61K 31/4045; A61K 31/4155; A61K 31/422; A61K 31/495; A61K 31/498; A61K 31/519; A61K 45/06

(57) ABSTRACT

The disclosure relates to methods and compositions for treating neurological conditions such as, such as, neurodegenerative disease, neuropathy, cerebral ischemia; and neurotrauma.

### BACKGROUND

The disclosure relates to the field of neurological disorders. Specifically, the disclosure relates to methods and compositions for treating neurological conditions such as, neurodegenerative disease, neuropathy, cerebral ischemia; and neurotrauma.

Neurological conditions are generally recognized to be diseases and conditions that result in, or are associated with, damage to neural cells/tissue. Such damage may be the result of, for example, the degeneration of, physical trauma to, and/or inflammation/oxidative stress within, a neural tissue. The clinical management of neurological conditions has been frustrated by the progressive nature of neurological diseases in addition to the limited efficacy and serious side-effects that are associated with the pharmacological agents currently available for treating these conditions. As such, conditions associated with as the degeneration of and/or injury/trauma to the nervous system have eluded most conventional pharmacological attempts to alleviate or cure these conditions.

Healthy proteostasis (protein synthesis and degradation) is a driver of organismal fitness, longevity, and decline in proteostasis is found as underlying mechanism of neurological conditions and aging with implications on the onset of age-related pathologies such as Alzheimer disease (AD). Protein aggregates are implicated in proteostasis-based cell fitness competition, an evolutionary-conserved process for maintaining adult tissue homeostasis and function. This process relies on the elimination of damaged, stressed and/or senescent cells by their healthy cell counterparts thus preventing their accumulation that would result in cell and tissue failure and the onset of chronic neurological disease.

Proteins that form protein aggregates can be disseminated with the circulation to distant organs explaining why some co-morbidities such as AD and type-2 diabetes are strongly linked. Community-based controlled studies and pathological analyses of autopsy derived brains and pancreases from within the same community indicate that T2D and prediabetes are more prevalent in AD than in non-AD control subjects. In T2D cases presenting with amyloid plaques, the duration of T2D correlated with the density of diffuse and neuritic plaques associated with AD.

New modalities for treating neurological conditions such as, neurodegenerative disease, neuropathy, cerebral ischemia; and neurotrauma are needed. The methods and compositions provided herein address these needs.

### BRIEF SUMMARY

The disclosure provides methods and compositions for treating neurological conditions such as, neurodegenerative disease, neuropathy, cerebral ischemia; and neurotrauma. Morae particularly, the disclosure provides methods of treating neurological conditions that comprise administering an effective amount of a HIF1-α Pathway Inhibitor or a HIF1-α Inhibitor and an PFKFB3 inhibitor to the subject having or at risk of having the neurological condition wherein the PFKFB3 Inhibitor does not inhibit the PI3K/AKT/mTOR pathway or HIF1-α.

Moreover, many neurological conditions such as AD show comparable histopathology to T2D, with amyloid protein aggregates derived from amyloid beta protein (Aβ) in the brain in AD and human islet amyloid pancreatic polypeptide (hIAPP) in the pancreas in T2D. Additionally, hIAPP and Aβ exert toxicity by similar mechanisms such as loss of calcium homeostasis, chronic injury and neuroinflammation. And Aβ and hIAPP protein aggregates share the induction of the same pathological cascade involving hypoxia-inducible factor-α (HIF1α) and PFKFB3 pathway. A key mechanism by which Aβ and hIAPP lead to the pathology seen in AD and T2D involves dysregulation of calcium homeostasis (in neurons and astrocytes, and β-cells in T2D), resulting in neuronal- and β-cell injury and decline. Aβ and hIAPP also trigger activation of microglia, leading to a neuroinflammatory response that further exacerbates AD and T2D.

Interestingly, in AD, not all of the damaged neurons with Aβ protein aggregates within an afflicted area die. There appear to be populations of neurons which survive the same conditions that kill their neighbours, since they become resistant to Aβ. The surviving neurons which are resistant to Aβ aggregates are dysfunctional and are marked by high expression levels of HIF1α and PFKFB3. Aβ-resistant neurons have increased aerobic glycolysis, and contribute to the cognitive impairments and dementia in AD. Not only Aβ-resistant neurons are glycolytic, adopting glycolytic metabolism via HIF1α-PFKFB3 activation, but also aerobic glycolysis is increased in the frontal and temporal cortex of patients with AD, indicating that Aβ-resistant neurons contribute to the impairment in the AD-affected brain regions. Thus, elevated HIF1α and PFKFB3 promote the survival of the Aβ stressed neurons at the expense of their neuronal function and correlate with the functional and cognitive impairment, memory loss and dementia associated with neurological conditions such as AD.

The inventors have surprisingly discovered that the combination of a HIF1a inhibitor and a PFKFB3 inhibitor is able to mitigate and possibly even reverse the damage caused by neurological conditions such as AD. Without being limited by theory, it is believed that in the context of AD, the disclosed methods result in neuronal regeneration after elimination of Aβ stressed and dysfunctional neurons by activated cell competition and thereby prevent or mitigate motor decline, functional and memory impairment and brain degeneration, and possibly even increasing neuronal regeneration and improving cognitive and memory function recovery.

The methods and compositions provided herein are useful in treating chronic and acute neurological conditions. In some embodiments, the disclosure provides m methods and compositions for treating a neurological condition selected from: a neurodegenerative disease such as Alzheimer's disease (AD), Parkinson's disease (PD), Huntington's disease (HD); dementia; mild cognitive impairment (MCI), aging-related memory impairment (AAMI), and neuropathy.

In some embodiments, the disclosure provides methods and compositions for treating an acute neurological condition. In further embodiments, the disclosure provides methods and compositions for treating a neurological condition selected from: stroke, neurotrauma (e.g., a traumatic brain injury or spinal cord injury), tethered spinal cord syndrome, cerebral ischemia, and global hypoxic ischemia.

In some embodiments, the disclosure provides:
[1] A method of treating a neurological condition in a subject in need thereof comprising:
   (a) Administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α.
[2] the method of [1], wherein the subject is administered an effective amount of the HIF1-α Pathway Inhibitor and the PFKFB3 inhibitor;
[3] the method of [1], wherein the subject is administered an effective amount of the HIF1-α Pathway Inhibitor and wherein the subject has previously been administered the PFKFB3 Inhibitor;
[4] the method of [1], wherein the subject is administered an effective amount of the PFKFB3 Inhibitor and wherein the subject has previously been administered the HIF1-α Pathway Inhibitor;
[5] the method of any one of [1]-[4], wherein the method of any one of 1(a)-1(c) is administered as a prophylactic treatment for the neurological condition;
[6] the method of any one of [1]-[4], wherein the subject has or is at risk of having the neurological condition;
[7] the method of any one of [1]-[4], wherein the subject has or has been diagnosed as having the neurological condition;
[8] the method of any one of [1]-[7], wherein the neurological condition is a neurodegenerative disease, dementia, neuropathy, or neurotrauma;
[9] the method of any one of [1]-[8], wherein the neurological condition is a neurodegenerative disease;
[10] the method of any one of [1]-[8], wherein the neurological condition is a dementia;
[11] the method of any one of [1]-[8], wherein the neurological condition is a neuropathy;
[12] the method of any one of [1]-[8], wherein the neurological condition is neurotrauma such as a spinal cord injury or a traumatic brain injury;
[13] the method of any one of [1]-[12], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor;
[14] the method of any one of [1]-[13], wherein the administered HIF1-α Pathway Inhibitor is silibinin, PX-478 or YC-1, or a salt thereof;
[15] the method of any one of [1]-[14], wherein the administered HIF1-α Pathway Inhibitor is ganetespib (ST-9090), phenethyl isothiocyanate, or BAY-87-2243, or a salt thereof;
[16] the method of any one of [1]-[15], wherein the administered HIF1-α Pathway Inhibitor is a HIF1-α Inhibitor;
[17] the method of [16], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[18] the method of [16] or [17], wherein the administered HIF1-α Inhibitor is antisense oligonucleotide EZN-2968 or nanobody AG-1, AG-2, AG-3, AG-4, AG-5, VHH212, or AHPC;
[19] the method of any one of [1]-[18], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[20] the method of any one of [1]-[19], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[21] the method of any one of [1]-[20], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[22] the method of any one of [1]-[21], wherein the HIF1-α Pathway Inhibitor and the PFKFB3 inhibitor are co-administered to the subject;
[23] the method of any one of [1]-[22], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[24] the method of any one of [1]-[23], wherein the HIF1-α Pathway Inhibitor and the PFKFB3 inhibitor are administered before the onset of one or more symptoms of the neurological condition;
[25] the method of any one of [1]-[24], wherein treating the neurological condition comprises delaying the onset of the neurological condition;
[26] the method of any one of [1]-[23], wherein the HIF1-α Pathway Inhibitor and the PFKFB3 inhibitor are administered after the onset of one or more symptoms of the neurological condition;
[27] the method of [26], wherein the method results in one or more symptoms of the neurological condition are reduced in the subject administered the HIF1-α Pathway Inhibitor and the PFKFB3 inhibitor compared to in the subject prior to treatment.
[28] the method of [27], wherein the one or more reduced symptoms of the neurological condition is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival;
[29] the method of [27] or [28], wherein the one or more symptoms of the neurological condition are reduced by at least 10%, at least 20%, at least 30%, at least 40%, or at least 50% compared to in the subject prior to treatment with the HIF1-α Pathway Inhibitor and the PFKFB3 inhibitor;
[30] the method according to any one of [27]-[29], wherein at least one of the behavioral reflexes; cognitive skills, balance, coordination, and cognitive skills of the subject are improved compared to in the subject prior to treatment with the HIF1-α Pathway Inhibitor and the PFKFB3 inhibitor;
[31] the method according to any one of [27]-[30], wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor;
[32] the method of any one of [1]-[3], which further comprises administering an additional Therapeutic agent to the subject;
[33] a method of treating a neurodegenerative disease in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[34] the method of [33], wherein the subject is administered an effective amount of the HIF1-α Pathway Inhibitor and the PFKFB3 inhibitor;
[35] the method of [33], wherein the subject is administered an effective amount of the HIF1-α Pathway Inhibitor and wherein the subject has previously been administered the PFKFB3 Inhibitor;
[36] the method of [33], wherein the subject is administered an effective amount of the PFKFB3 Inhibitor and wherein the subject has previously been administered the HIF1-α Pathway Inhibitor;
[37] the method of any one of [33]-[36], wherein the method of any one of 1(a)-1(c) is administered as a prophylactic treatment for the neurodegenerative disease;
[38] the method of any one of [33]-[36], wherein the subject has or is at risk of having the neurodegenerative disease;
[39] the method of any one of [33]-[36], wherein the subject has or has been diagnosed as having the neurodegenerative disease;
[40] the method of any one of [33]-[39], wherein the neurodegenerative disease is selected from Alzheimer's disease (AD), Parkinson's disease (PD), Huntington's disease (HD), amyotrophic lateral sclerosis (ALS), Friedreich's ataxia, frontotemporal lobar degeneration, or dementia with;
[41] the method of [40], wherein the neurodegenerative disease is Alzheimer's disease (AD);
[42] the method of [40], wherein the neurodegenerative disease is a Parkinson's disease (PD);
[43] the method of [40], wherein the neurodegenerative disease is a Huntington's disease (HD);
[44] the method of [40], wherein the neurodegenerative disease is a amyotrophic lateral sclerosis (ALS);
[45] the method of any one of [33]-[44], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[46] the method of any one of [33]-[45], wherein the administered HIF1-α Pathway Inhibitor is silibinin, PX-478 or YC-1, or a salt thereof;
[47] the method of any one of [33]-[45], wherein the administered HIF1-α Pathway Inhibitor is ganetespib (ST-9090), phenethyl isothiocyanate, or BAY-87-2243, or a salt thereof;
[48] the method of any one of [33]-[47], wherein the administered HIF1-α Pathway Inhibitor is a HIF1-α Inhibitor;
[49] the method of [48], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[50] the method of [48 or 49], wherein the administered HIF1-α Inhibitor is Antisense oligonucleotide EZN-2968 or nanobody AG-1, AG-2, AG-3, AG-4, AG-5, VHH212, or AHPC;
[51] the method of any one of [33]-[50], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[52] the method of any one of [33]-[51], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[53] the method of any one of [33]-[51], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[54] the method of any one of [33]-[53], wherein the HIF1-α Pathway Inhibitor and the PFKFB3 inhibitor are co-administered to the subject;
[55] the method of any one of [33]-[54], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.
[56] the method of any one of [33]-[55], wherein the HIF1-α Pathway Inhibitor and the PFKFB3 inhibitor are administered before the onset of one or more symptoms of the neurodegenerative disease.
[57] the method of any one of [33]-[56], wherein treating the neurodegenerative disease comprises delaying the onset of the neurodegenerative disease.
[58] the method of any one of [33]-[57], wherein the HIF1-α Pathway Inhibitor and the PFKFB3 inhibitor are administered after the onset of one or more symptoms of the neurodegenerative disease.
[59] the method of any one of [33]-[58], wherein the method results in reduction in one or more symptoms of the neurodegenerative disease the subject administered the HIF1-α Pathway Inhibitor and the PFKFB3 inhibitor compared to in the subject prior to treatment.
[60] the method of [59], wherein the one or more reduced symptoms of the neurodegenerative disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time;
[61] the method of [59] or [60], wherein the one or more symptoms of the neurodegenerative disease are reduced by at least 10%, at least 20%, at least 30%, at least 40%, or at least 50% compared to in the subject prior to treatment with the HIF1-α Pathway Inhibitor and the PFKFB3 inhibitor.
[62] the method according to any one of [59]-[61], wherein at least one of the behavioral reflexes; cognitive skills, balance, coordination, and cognitive skills of the subject are improved compared to in the subject prior to treatment with the HIF1-α Pathway Inhibitor and the PFKFB3 inhibitor;
[63] the method according to any one of [59]-[62], wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor;
[64] the method of any one of [33]-[63], which further comprises administering an additional Therapeutic agent to the subject.
[65] a method of treating Alzheimer's disease (AD) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α.
[66] the method of [65], wherein the subject is administered an effective amount of the HIF1-α Pathway Inhibitor and the PFKFB3 inhibitor;
[67] the method of [65], wherein the subject is administered an effective amount of the HIF1-α Pathway Inhibitor and wherein the subject has previously been administered the PFKFB3 Inhibitor;
[68] the method of [65], wherein the subject is administered an effective amount of the PFKFB3 Inhibitor and wherein the subject has previously been administered the HIF1-α Pathway Inhibitor;
[69] the method of any one of [65]-[68], wherein the method of any one of 1(a)-1(c) is administered as a prophylactic treatment for Alzheimer's disease;
[70] the method of any one of [65]-[68], wherein the subject has or is at risk of having Alzheimer's disease;
[71] the method of any one of [65]-[68], wherein the subject has or has been diagnosed as having Alzheimer's disease;
[72] the method of any one of [65]-[71], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[73] the method of any one of [65]-[72], wherein the administered HIF1-α Pathway Inhibitor is silibinin, PX-478 or YC-1, or a salt thereof;
[74] the method of any one of [65]-[72], wherein the administered HIF1-α Pathway Inhibitor is ganetespib (ST-9090), phenethyl isothiocyanate, or BAY-87-2243, or a salt thereof;
[75] the method of any one of [65]-[72], wherein the administered HIF1-α Pathway Inhibitor is a HIF1-α Inhibitor.
[76] the method of [75], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[77] the method of [75] or [76], wherein the administered HIF1-α Inhibitor is Antisense oligonucleotide EZN-2968 or nanobody AG-1, AG-2, AG-3, AG-4, AG-5, VHH212, or AHPC;
[78] the method of any one of [65]-[77], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[79] the method of any one of [65]-[78], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[80] the method of any one of [65]-[78], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[81] the method of any one of [65]-[80], wherein the HIF1-α Pathway Inhibitor and the PFKFB3 inhibitor are co-administered to the subject;
[82] the method of any one of [65]-[81], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.
[83] the method of any one of [65]-[82], wherein the HIF1-α Pathway Inhibitor and the PFKFB3 inhibitor are administered before the onset of one or more symptoms of Alzheimer's disease.
[84] the method of any one of [65]-[83], wherein treating Alzheimer's disease comprises delaying the onset of Alzheimer's disease.
[85] the method of any one of [65]-[84], wherein the HIF1-α Pathway Inhibitor and the PFKFB3 inhibitor are administered after the onset of one or more symptoms of Alzheimer's disease.
[86] the method of any one of [65]-[85], wherein the method results in reduction in one or more symptoms of Alzheimer's disease the subject administered the HIF1-α Pathway Inhibitor and the PFKFB3 inhibitor compared to in the subject prior to treatment.
[87] the method of [86], wherein the one or more reduced symptoms of Alzheimer's disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival;
[88] the method of [86] or [87], wherein the one or more symptoms of Alzheimer's disease are reduced by at least 10%, at least 20%, at least 30%, at least 40%, or at least 50% compared to in the subject prior to treatment with the HIF1-α Pathway Inhibitor and the PFKFB3 inhibitor.
[89] the method according to any one of [86]-[88], wherein at least one of the behavioral reflexes; balance, coordination, and cognitive skills of the subject are improved compared to in the subject prior to treatment with the HIF1-α Pathway Inhibitor and the PFKFB3 inhibitor;
[90] the method according to any one of [86]-[89], wherein at least one of the cognitive skills of the subject are improved by at least 10%, at least 20%, at least 30%, at least 40%, or at least 50%, or at least one of the following symptoms are reduced in the subject compared to in the subject prior to treatment with the HIF1-α Pathway Inhibitor and the PFKFB3 inhibitor: forgetfulness, difficulty solving simple math problems; trouble remembering how to do simple tasks; inability to think clearly; difficulty speaking, understanding, reading, or writing; confusion, irritability, mood swings, anxiety, aggressiveness, or tendency to wander away from home;
[91] the method of any one of [65]-[90], which further comprises administering an additional Therapeutic agent to the subject;
[92] a method of treating Parkinson's disease (PD) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[93] the method of [92], wherein the subject is administered an effective amount of the HIF1-α Pathway Inhibitor and the PFKFB3 inhibitor;
[94] the method of [92], wherein the subject is administered an effective amount of the HIF1-α Pathway Inhibitor and wherein the subject has previously been administered the PFKFB3 Inhibitor;
[95] the method of [92], wherein the subject is administered an effective amount of the PFKFB3 Inhibitor and wherein the subject has previously been administered the HIF1-α Pathway Inhibitor;
[96] the method of any one of [92]-[95], wherein the method of any one of [92(a)-(c)] is administered as a prophylactic treatment for Parkinson's disease;
[97] the method of any one of [92]-[95], wherein the subject has or is at risk of having Parkinson's disease;
[98] the method of any one of [92]-[95], wherein the subject has or has been diagnosed as having Parkinson's disease;
[99] the method of any one of [92]-[98], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[100] the method of any one of [92]-[99], wherein the administered HIF1-α Pathway Inhibitor is silibinin, PX-478 or YC-1, or a salt thereof;
[101] the method of any one of [92]-[99], wherein the administered HIF1-α Pathway Inhibitor is ganetespib (ST-9090), phenethyl isothiocyanate, or BAY-87-2243, or a salt thereof;
[102] the method of any one of [92]-[99], wherein the administered HIF1-α Pathway Inhibitor is a HIF1-α Inhibitor;
[103] the method of [102], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[104] the method of [102] or [103], wherein the administered HIF1-α Inhibitor is Antisense oligonucleotide EZN-2968 or nanobody AG-1, AG-2, AG-3, AG-4, AG-5, VHH212, or AHPC;
[105] the method of any one of [92]-[104], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[106] the method of any one of [92]-[105], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[107] the method of any one of [92]-[105], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[108] the method of any one of [92]-[107], wherein the HIF1-α Pathway Inhibitor and the PFKFB3 inhibitor are co-administered to the subject;
[109] the method of any one of [92]-[108], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[110] the method of any one of [92]-[109], wherein the HIF1-α Pathway Inhibitor and the PFKFB3 inhibitor are administered before the onset of one or more symptoms of Parkinson's disease;
[111] the method of any one of [92]-[110], wherein treating Parkinson's disease comprises delaying the onset of Parkinson's disease;
[112] the method of any one of [92]-[111], wherein the HIF1-α Pathway Inhibitor and the PFKFB3 inhibitor are administered after the onset of one or more symptoms of Parkinson's disease;
[113] the method of any one of [92]-[112], wherein the method results in reduction in one or more symptoms of Parkinson's disease the subject administered the HIF1-α Pathway Inhibitor and the PFKFB3 inhibitor compared to in the subject prior to treatment;
[114] the method of [113], wherein the one or more reduced symptoms of Parkinson's disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival;
[115] the method of [113 or 114], wherein the one or more symptoms of Parkinson's disease are reduced by at least 10%, at least 20%, at least 30%, at least 40%, or at least 50% compared to in the subject prior to treatment with the HIF1-α Pathway Inhibitor and the PFKFB3 inhibitor;
[116] the method according to any one of [113]-[115], wherein at least one of the following symptoms is improved in the subject compared to prior to treatment with the HIF1-α Pathway Inhibitor and the PFKFB3 inhibitor: tremors slowed movement (bradykinesia), rigid muscles, impaired posture and balance; loss of automatic movements, changes in speech, or difficulty writing;
[117] the method according to any one of [113]-[116], wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor;
[118] the method of any one of [92]-[117], which further comprises administering an additional Therapeutic agent to the subject;
[119] a method of treating Huntington's disease (HD) in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[120] the method of [119], wherein the subject is administered an effective amount of the HIF1-α Pathway Inhibitor and the PFKFB3 inhibitor;
[121] the method of [119], wherein the subject is administered an effective amount of the HIF1-α Pathway Inhibitor and wherein the subject has previously been administered the PFKFB3 Inhibitor;
[122] the method of [119], wherein the subject is administered an effective amount of the PFKFB3 Inhibitor and wherein the subject has previously been administered the HIF1-α Pathway Inhibitor;
[123] the method of any one of [119]-[122], wherein the method of any one of [119(a)-(c)] is administered as a prophylactic treatment for Huntington's disease;
[124] the method of any one of [119]-[122], wherein the subject has or is at risk of having Huntington's disease;
[125] the method of any one of [119]-[122], wherein the subject has or has been diagnosed as having Huntington's disease;
[126] the method of any one of [119]-[125], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[127] the method of any one of [119]-[126], wherein the administered HIF1-α Pathway Inhibitor is silibinin, PX-478 or YC-1, or a salt thereof;
[128] the method of any one of [119]-[126], wherein the administered HIF1-α Pathway Inhibitor is ganetespib (ST-9090), phenethyl isothiocyanate, or BAY-87-2243, or a salt thereof;
[129] the method of any one of [119]-[126], wherein the administered HIF1-α Pathway Inhibitor is a HIF1-α Inhibitor;
[130] the method of [129], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[131] the method of [129 or 130], wherein the administered HIF1-α Inhibitor is Antisense oligonucleotide EZN-2968 or nanobody AG-1, AG-2, AG-3, AG-4, AG-5, VHH212, or AHPC;
[132] the method of any one of [119]-[131], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[133] the method of any one of [119]-[132], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[134] the method of any one of [119]-[132], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[135] the method of any one of [119]-[134], wherein the HIF1-α Pathway Inhibitor and the PFKFB3 inhibitor are co-administered to the subject;
[136] the method of any one of [119]-[135], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[137] the method of any one of [119]-[136], wherein the HIF1-α Pathway Inhibitor and the PFKFB3 inhibitor are administered before the onset of one or more symptoms of Huntington's disease;
[138] the method of any one of [119]-[137], wherein treating Huntington's disease comprises delaying the onset of Huntington's disease;
[139] the method of any one of [119]-[138], wherein the HIF1-α Pathway Inhibitor and the PFKFB3 inhibitor are administered after the onset of one or more symptoms of Huntington's disease;
[140] the method of any one of [119]-[139], wherein the method results in reduction in one or more symptoms of Huntington's disease the subject administered the HIF1-α Pathway Inhibitor and the PFKFB3 inhibitor compared to in the subject prior to treatment;
[141] the method of [140], wherein the one or more reduced symptoms of Huntington's disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time;
[142] the method of [140] or [141], wherein the one or more symptoms of Huntington's disease are reduced by at least 10%, at least 20%, at least 30%, at least 40%, or at least 50% compared to in the subject prior to treatment with the HIF1-α Pathway Inhibitor and the PFKFB3 inhibitor;
[143] the method according to any one of [140]-[142], wherein at least one of the cognitive skills of the subject are improved by at least 10%, at least 20%, at least 30%, at least 40%, or at least 50%, or at least one of the following symptoms are reduced in the subject compared to in the subject prior to treatment with the HIF1-α Pathway Inhibitor and the PFKFB3 inhibitor: memory impairment, confusion, impaired judgement slurred speech, difficulty problem solving, personality changes, depression, mood swings, unsteady gait, involuntary chorea, twitching and jerking movements and tremors, and dementia;
[144] the method according to any one of [140]-[143], wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor;
[145] the method of any one of [119]-[144], which further comprises administering an additional Therapeutic agent to the subject;
[146] a method of treating neurotrauma in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[147] the method of [146], wherein the subject is administered an effective amount of the HIF1-α Pathway Inhibitor and the PFKFB3 inhibitor;
[148] the method of [146], wherein the subject is administered an effective amount of the HIF1-α Pathway Inhibitor and wherein the subject has previously been administered the PFKFB3 Inhibitor;
[149] the method of [146], wherein the subject is administered an effective amount of the PFKFB3 Inhibitor and wherein the subject has previously been administered the HIF1-α Pathway Inhibitor;
[150] the method of any one of [146]-[149], wherein the method of any one of [146(a)-(c)] is administered as a prophylactic treatment for neurotrauma;
[151] the method of any one of [146]-[149], wherein the subject has or is at risk of having neurotrauma;
[152] the method of any one of [146]-[149], wherein the subject has or has been diagnosed as having neurotrauma;
[153] the method of any one of [146]-[153], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[154] the method of any one of [146]-[153], wherein the administered HIF1-α Pathway Inhibitor is silibinin, PX-478 or YC-1, or a salt thereof;
[155] the method of any one of [146]-[153], wherein the administered HIF1-α Pathway Inhibitor is ganetespib (ST-9090), phenethyl isothiocyanate, or BAY-87-2243, or a salt thereof;
[156] the method of any one of [146]-[153], wherein the administered HIF1-α Pathway Inhibitor is a HIF1-α Inhibitor;
[157] the method of [156], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[158] the method of [156] or [157], wherein the administered HIF1-α Inhibitor is Antisense oligonucleotide EZN-2968 or nanobody AG-1, AG-2, AG-3, AG-4, AG-5, VHH212, or AHPC;
[159] the method of any one of [146]-[158], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[160] the method of any one of [146]-[159], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[161] the method of any one of [146]-[159], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[162] the method of any one of [146]-[161], wherein the HIF1-α Pathway Inhibitor and the PFKFB3 inhibitor are co-administered to the subject;
[163] the method of any one of [146]-[162], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[164] the method of any one of [146]-[163], wherein the neurotrauma is spinal cord injury;
[165] the method of any one of [146]-[163], wherein the neurotrauma is traumatic brain injury;
[166] the method of any one of [146]-[165], wherein the HIF1-α Pathway Inhibitor and the PFKFB3 inhibitor are administered before the onset of one or more symptoms of neurotrauma;
[167] the method of any one of [146]-[165], wherein the HIF1-α Pathway Inhibitor and the PFKFB3 inhibitor are administered before the onset of one or more symptoms of neurotrauma;
[168] the method of any one of [146]-[167], wherein treating neurotrauma comprises delaying the onset of neurotrauma;
[169] the method of any one of [146]-[168], wherein the HIF1-α Pathway Inhibitor and the PFKFB3 inhibitor are administered after the onset of one or more symptoms of neurotrauma;
[170] the method of any one of [146]-[169], wherein the method results in reduction in one or more symptoms of neurotrauma the subject administered the HIF1-α Pathway Inhibitor and the PFKFB3 inhibitor compared to in the subject prior to treatment;
[171] the method of [170], wherein the one or more reduced symptoms of neurotrauma is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time;
[172] the method of [170] or [171], wherein the one or more symptoms of neurotrauma are reduced by at least 10%, at least 20%, at least 30%, at least 40%, or at least 50% compared to in the subject prior to treatment with the HIF1-α Pathway Inhibitor and the PFKFB3 inhibitor;
[173] the method according to any one of [170]-[172], wherein at least one of the behavioral reflexes; balance, coordination, and cognitive skills of the subject are improved compared to in the subject prior to treatment with the HIF1-α Pathway Inhibitor and the PFKFB3 inhibitor;
[174] the method according to any one of [170]-[173], wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor; and/or
[175] the method of any one of [146]-[174], which further comprises administering an additional Therapeutic agent to the subject.

### BRIEF DESCRIPTION OF THE DRAWINGS/FIGURES

FIGS. 1A-1E, depict exemplary PFKFB3 small molecule inhibitors.

### DETAILED DESCRIPTION

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Other features and advantages of the disclosed methods and compositions will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, include plural forms unless it is expressly stated or is unambiguously clear from the context that such is not intended. The singular form “a”, “an”, and “the” also includes the statistical mean composition, characteristics, or size of the particles in a population of particles (e.g., mean polyethylene glycol molecular weight mean liposome diameter, mean liposome zeta potential). The mean particle size and zeta potential of liposomes in a pharmaceutical composition can routinely be measured using methods known in the art, such as dynamic light scattering. The mean amount of a therapeutic agent in a nanoparticle composition may routinely be measured for example, using absorption spectroscopy (e.g., ultraviolet-visible spectroscopy).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean +/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Recitation of ranges of values herein are merely intended to serve as a shorthand method of referring individually to each separate value falling within the range, unless otherwise indicated herein, and each separate value is incorporated into the specification as if it were individually recited herein.

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed methods and compositions also envisage the explicit exclusion of one or more of any of the group members in the disclosed compositions or methods.

The terms “antibody” and “antigen-binding antibody fragment” and the like, as used herein, include any protein or peptide containing molecule that comprises at least a portion of an immunoglobulin molecule, such as, but not limited to, at least one complementarity determining region (CDR) of a heavy or light chain or an antigen binding portion thereof.

The term “antibody” also includes fragments, specified portions and variants thereof, including antibody mimetics or comprising portions of antibodies that mimic the structure and/or function of an antibody or specified fragment or portion thereof, including single chain antibodies, single binding domain antibodies and antigen binding antibody fragments.

The term “antibody fragment” refers to a portion of an intact antibody, generally the antigen binding or variable region of an intact antibody. Examples of antibody fragments include, but are not limited to Fab, Fab′, F(ab′)2, single chain (scFv) and Fv fragments, diabodies; linear antibodies; single-chain antibody molecules; single Fab arm “one arm” antibodies and multispecific antibodies formed from antibody fragments, among others. Antibody fragments include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an antigen or antigen receptor or binding protein, which can be incorporated into an antibody provided herein.

Antibody fragments can be produced by enzymatic cleavage, synthetic or recombinant techniques, as known in the art. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The terms “nucleic acid” and “oligonucleotide” are used interchangeably herein and refer to at least two nucleotides covalently linked together. In some embodiments the HIF1-α pathway inhibitor and/or PFKFB3 inhibitor administered according to the provided methods is a therapeutic nucleic acid. In some embodiments, the administered nucleic acid is an ENMD-1198, an shRNA, a Dicer substrate (e.g., dsRNA), an miRNA, an anti-miRNA, an antisense molecule, a decoy, or an aptamer, or a plasmid capable of expressing a ENMD-1198, an shRNA, a Dicer substrate, an miRNA, an anti-miRNA, an antisense molecule, a decoy, or an aptamer.

The nucleic acids administered according to the provided methods are preferably single-stranded or double-stranded and generally contain phosphodiester bonds, although in some cases, nucleic acid/oligonucleotide analogs are included that have alternate backbones, comprising, for example, phosphoramide, phosphorothioate, phosphorodithioate, O-methylphosphoroamidiate linkages, and peptide nucleic acid backbones and linkages. Other analog nucleic acids/oligonucleotides include those with positive backbones; non-ionic backbones, and non-ribose backbones. Nucleic acids/oligonucleotides containing one or more carbocyclic sugars are also included within the definition of nucleic acids and oligonucleotides. These modifications of the ribose-phosphate backbone may be done for example, to facilitate the addition of additional moieties such as labels, or to increase the stability and half-life of such molecules in physiological environments. Nucleic acid/oligonucleotide backbones of oligonucleotides used according to the provided methods can range from about 5 nucleotides to about 750 nucleotides. Preferred nucleic acid/oligonucleotide backbones range from about 5 nucleotides to about 500 nucleotides, and preferably from about 10 nucleotides to about 100 nucleotides in length.

The oligonucleotides administered according to the provided methods are polymeric structures of nucleoside and/or nucleotide monomers capable of specifically hybridizing to at least a region of a nucleic acid target. As indicated above, the “nucleic acids” and “oligonucleotides” used according to the provided methods include, but are not limited to, compounds comprising naturally occurring bases, sugars and intersugar (backbone) linkages, non-naturally occurring modified monomers, or portions thereof (e.g., oligonucleotide analogs or mimetics) which function similarly to their naturally occurring counterpart, and combinations of these naturally occurring and non-naturally occurring monomers. As used herein, the term “modified” or “modification” includes any substitution and/or any change from a starting or natural oligomeric compound, such as an nucleic acid. Modifications to nucleic acids encompass substitutions or changes to internucleoside linkages, sugar moieties, or base moieties, such as those described herein and those otherwise known in the art.

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

The terms “condition” and “disease” are used interchangeably herein.

The term “neurological condition” as used herein generally refers to a disease or condition resulting in, or associated with, damage to neural cells/tissue. Such damage may be the result of, for example, the degeneration of, physical trauma to, and/or inflammation/oxidative stress within a neural tissue. The term “progression of a neurological condition” refers to the gradual worsening of the disease over time, whereby symptoms and neurochemical deficits become increasingly more debilitating and/or intense. The progression of neurological condition often correlates to a decline in the structure, activity, and/or function of brain tissue. As used herein, the term “inhibiting progression of a neurological condition” refers to slowing and/or stopping the progression of symptoms and neurochemical deficits of a neurological condition.

In another embodiment, the method of inhibiting progression of a neurological conditions further comprises administering a second active pharmaceutical ingredient effective for the treatment of the neurological condition.

As used herein, the term “neurodegenerative disease” refers to a disease characterized by a progressive decline in the structure, activity, and/or function of neural tissue, including brain tissue. Exemplary neurodegenerative disease that can be treated according to the provided methods include, but are not limited to, Alzheimer's disease (AD), Parkinson's disease (PD), Huntington's disease (HD), amyotrophic lateral sclerosis (ALS), Friedreich's ataxia, frontotemporal lobar degeneration, and dementia (e.g., AD-type senile dementia (SDAT), vascular dementia, or dementia with Lewy bodies).

As used herein, the term “progression of a neurodegenerative disease” refers to the gradual worsening of the disease over time, whereby symptoms and neurochemical deficits become increasingly more debilitating and/or intense. Neurodegenerative disease progression often correlates to a decline in the structure, activity, and/or function of brain tissue. The term “inhibiting progression of a neurodegenerative disease” refers to slowing and/or stopping the progression of symptoms and/or neurochemical deficits of a neurodegenerative disease. As used herein, “delaying development” of a neurological condition, such as AD, PD, HD, ALS, and dementia means to defer, hinder, slow, retard, stabilize, and/or postpone development of one or more symptoms, of the condition, including decreasing the rate at which the subject's disease progresses (e.g., to shift the subject from rapidly progressing disease to a more slowly progressing disease). This delay can be of varying lengths of time, depending on the history of the condition and/or the medical profile of the subject being treated. A sufficient or significant delay can, in effect, encompass prevention, in that the subject does not develop detectable disease. A method that “delays” development of a condition is a method that reduces the extent of the condition in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations of the neurological condition are lessened and/or the time course of the progression is slowed or lengthened, as compared to not administering the agent. Thus, “delaying development” also includes, but is not limited to, alleviation of symptoms, diminishment of extent of t, stabilized (i.e., not worsening) state of disease, delay or slowing of disease progression, and remission (whether partial or total) whether detectable or undetectable.

The terms “neurotrauma”, “neural trauma”, and “neural injury” are used interchangeably herein and refer to mechanical injury to the brain or spinal cord. The terms “damage caused by neurotrauma” or “neurotrauma-induced damage” refer to damage caused by a mechanical injury to the brain or spinal cord.

Reference to spinal cord injury (SCI) herein includes any form of physical, chemical or genetic trauma to the spinal cord. A physical trauma includes a tissue insult such as an abrasion, incision, contusion, puncture, compression etc., such as can arise from traumatic contact of a foreign object with any locus of or appurtenant/adjacent to the head, neck or vertebral column. Other forms of traumatic injury can arise from constriction or compression of central nervous system (CNS) tissue by an inappropriate accumulation of fluid (e.g., a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor or from disease (e.g., poliomyelitis, spina bifida, Friedreich's Ataxia, etc.). In some embodiments, the methods and compositions provided herein are useful for treating or preventing secondary injury resulting from an initial insult/injury to the CNS (e.g., spinal cord).

As used herein, the term “stroke” refers to the sudden death of brain cells due to a lack of oxygen when the blood flow to the brain is impaired by blockage or rupture of an artery to the brain. Risk factors associated with an increased likelihood of having a stroke include old age, high blood pressure, previous stroke or transient ischemic attack, diabetes, high cholesterol, smoking and atrial fibrillation.

As used herein an “effective amount” refers to a dosage of an agent sufficient to provide a medically desirable prophylactic and/or therapeutic effect on a neurological condition (e.g., a neuropathy, a neurodegenerative disorder such as AD, PD, HD, ALS, dementia, cerebral ischemia, or a neural injury such as a spinal cord injury) or on a neural cell and/or tissue (e.g., CNS tissue, such as brain or spinal cord). The effective amount will vary with the desired outcome, the particular neurological condition being treated (or prevented), the age and physical condition of the subject being treated, the severity of the condition, the duration of the treatment, the nature of the concurrent or combination therapy (if any), the specific route of administration and like factors within the knowledge and expertise of the health practitioner.

An “effective amount” can be determined empirically and in a routine manner, in relation to the stated purpose. A prophylactic and/or therapeutic effect includes, but is not limited to, reduction in apoptosis/destruction/loss of the number and/or function of neural cells and/or tissue; increase survival of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement in cognitive function; and increased survival/survival time.

The terms “subject”, “patient,” “individual,” and “animal” are used interchangeably and refer to mammals such as human patients and non-human primates, as well as experimental animals such as rabbits, rats, and mice, and other laboratory animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

Terms such as “treating,” or “treatment,” “to treat,” or “therapy,” refer to both (a) therapeutic measures that cure, slow down, attenuate, lessen symptoms of, and/or halt progression of a pathologic condition and (b) prophylactic or preventative measures that prevent and/or slow the development of a targeted condition and or its related symptoms

Thus, subjects in need of treatment include those already with the neurological condition; those at risk of having the neurological condition; and those in whom the neurological condition is to be prevented. Subjects can routinely be identified as “having or at risk of having” a neurological condition or another condition referred to herein using medical and diagnostic techniques known in the art. In certain embodiments, a subject is successfully “treated” according to the provided methods if the subject shows, e.g., total, partial, or transient amelioration or elimination of a symptom associated with the condition (e.g., a neurodegenerative disorder such as AD, PD, HD, ALS or dementia, or a neural injury such as a spinal cord injury). In specific embodiments, the terms “treating,” or “treatment,” “to treat,” or “therapy” refer to the amelioration of at least one measurable physical parameter of an neurological condition, such as improvement of cognitive symptoms or protection of nerve cells. In other embodiments, the terms “treating,” or “treatment,” “to treat,” or “therapy,” refer to the inhibition of the progression of a neurological condition, either physically by, e.g., stabilization of a discernible symptom, physiologically by, e.g., stabilization of a physical parameter, or both. In other embodiments, the terms “treating,” or “treatment,” “to treat,” or “therapy,” refer to the alleviation of symptoms, the reduction of inflammation, the inhibition of cell death, and/or the restoration of cell function. Treatment can be with the HIF1-α Pathway Inhibitor and PFKFB3 inhibitor compositions disclosed herein, or in further combination with one or more additional Therapeutic agent.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, carrier, excipient, stabilizer, diluent, or preservative. Pharmaceutically acceptable carriers can include for example, one or more compatible solid or liquid filler, diluents or encapsulating substances which are suitable for administration to a human or other subject.

“Therapeutic agent(s)” used according to the disclosed methods and compositions can additionally include any agent directed to treat a condition in a subject.

### PFKFB3 Inhibitors

PFKFB3 (6-phosphofructo-2-kinase-fructose-2,6-bisphosphatase 3) is a bifunctional protein that is involved in both the synthesis and degradation of fructose-2,6-bisphosphate, a regulatory molecule that controls glycolysis in eukaryotes and is required for cell cycle progression and the prevention of apoptosis.

In some embodiments, the disclosure provides a method of treating a neurological condition in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor to the subject;
   wherein the PFKFB3 Inhibitor does not inhibit the PI3K/AKT/mTOR pathway or HIF1-α.

The PFKFB3 Inhibitors that can be used according to the provided methods are not particularly limited. In some embodiments, the administered PFKFB3 Inhibitor is an antibody or a PFKFB3-binding antibody (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, miRNA, dsRNA, ssRNA, and shRNA), a peptibody, a nanobody, a PFKFB3 inhibitory binding polypeptide, or a small molecule PFKFB3 Inhibitor.

In some embodiments, the PFKFB3 inhibitor administered according to the provide methods has an IC50 for a PFKFB3 activity/function of 100 μM or lower concentration for a PFKFB3 activity. In some embodiments, the PFKFB3 inhibitor has an IC50 of at least or at most or about 200, 100, 80, 50, 40, 20, 10, 5, or 1 μM, or at least or at most or about 100, 10, or 1 nM, or lower (or any range or value derivable therefrom). In some embodiments, the PFKFB3 inhibitor inhibits the expression of PFKFB3. Assays for determining the ability of a compound to inhibit PFKFB3 activity are known in the art. In some embodiments, the inhibition of PFKFB3 activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit PFKFB3 activity.

In some embodiments, the PFKFB3 Inhibitor administered according to the provided methods is an antibody or a PFKFB3-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), In particular embodiments, the administered PFKFB3 Inhibitor is a nanobody (e.g., a VHH).

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, miRNA, dsRNA, ssRNA, and shRNA). In particular embodiments, the HIF1-α Inhibitor administered according to the provided methods is an siRNA or an antisense oligonucleotide.

Representative examples of human PFKFB3 coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit PFKFB3 activity can routinely be designed and prepared based on each of the above human PFKFB3 transcript sequences using methods known in the art.

The administration of PFKFB3 inhibitory nucleic acids or any ways of inhibiting gene expression of PFKFB3 known in the art are contemplated in certain embodiments of the provided methods. Examples of inhibitory nucleic acid include but are not limited to, antisense nucleic acids such as: ENMD-1198 (small interfering RNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of PFKFB3 or prevent the translation of a PFKFB3 gene transcript in a cell. In some embodiments, the PFKFB3 inhibitory nucleic acid administered according to the provided methods is from 16 to 1000 nucleotides in length. In certain embodiments the administered PFKFB3 inhibitory nucleic acid is from 18 to 100 nucleotides long. In certain embodiments the administered PFKFB3 inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the PFKFB3 inhibitory nucleic acid administered according to the provided methods is capable of decreasing the expression of PFKFB3 by at least 10%, 20%, 30%, or 40%, more particularly by at least 50%, 60%, or 70%, and most particularly by at least 75%, 80%, 90%, 95% or more or any range or value in between the foregoing.

In some embodiments, the PFKFB3 inhibitory nucleic acid administered according to the provided methods is between 17 to 25 nucleotides in length and comprises a 5′ to 3′ sequence that is at least 90% complementary to the 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). In some embodiments, the administered PFKFB3 inhibitory nucleic acid is 17, 18, 19, 20, 21, 22, 23, 24, or 25 nucleotides in length, or any range derivable therein. In some embodiments, the administered PFKFB3 inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the PFKFB3 inhibitory nucleic acid administered according to the provided methods is a miRNA. In further embodiments, the administered miRNA is a member selected from: hsa-mir-26b-5p (MIRT028775), hsa-mir-330-3p (MIRT043840), hsa-mir-6779-5p (MIRT454747), hsa-mir-6780a-5p (MIRT454748), hsa-mir-3689c (MIRT454749), hsa-mir-3689b-3p (MIRT454750), hsa-mir-3689a-3p (MIRT454751), hsa-mir-30b-3p (MIRT454752), hsa-mir-1273h-5p (MIRT454753), hsa-mir-6778-5p (MIRT454754), hsa-mir-1233-5p (MIRT454755), hsa-mir-6799-5p (MIRT454756), hsa-mir-7106-5p (MIRT454757), hsa-mir-6775-3p (MIRT454758), hsa-mir-1291 (MIRT454759), hsa-mir-765 (MIRT454760), hsa-mir-423-5p (MIRT454761), hsa-mir-3184-5p (MIRT454762), hsa-mir-6856-5p (MIRT454763), hsa-mir-6758-5p (MIRT454764), hsa-mir-3185 (MIRT527973), hsa-mir-6892-3p (MIRT527974), hsa-mir-6840-5p (MIRT527975), and hsa-mir-6865-3p (MIRT527976).

In some embodiments, the PFKFB3 inhibitor administered according to the provide methods is a small molecule. The administered small molecule PFKFB3 inhibitors may be any small molecules that is determined to inhibit PFKFB3 function or activity. Such small molecules may be determined based on functional assays in vitro or in vivo.

In some embodiments, the PFKFB3 inhibitor small molecules administered according to the provide methods is a small molecule PFKFB3 inhibitory molecules disclosed in U.S. publication nos. 20130059879, 20120177749, 20100267815, 20100267815, and 20090074884, the disclosure of each of which is herein incorporated by reference in its entirety.

In some embodiments, the PFKFB3 inhibitor administered according to the provided methods is at least one of: (1H-Benzo[g]indol-2-yl)-phenyl-methanone; (3H-Benzo[e]indol-2-yl)-phenyl-methanone; (3H-Benzo[e]indol-2-yl)-(4-methoxy-phenyl)-methanone; (3H-Benzo[e]indol-2-yl)-pyridin-4-yl-methanone; HCl salt of (3H-Benzo[e]indol-2-yl)-pyridin-4-yl-methanone; (3H-Benzo[e]indol-2-yl)-(3-methoxy-phenyl)-methanone; (3H-Benzo[e]indol-2-yl)-pyridin-3-yl-methanone; (3H-Benzo[e]indol-2-yl)-(2-methoxy-phenyl)-methanone; (3H-Benzo[e]indol-2-yl)-(2-hydroxy-phenyl)-methanone; (3H-Benzo[e]indol-2-yl)-(4-hydroxy-phenyl)-methanone; (5-Methyl-3H-benzo[e]indol-2-yl)-phenyl-methanone; Phenyl-(7H-pyrrolo[2,3-h]quinolin-8-yl)-methanone; (3H-Benzo[e]indol-2-yl)-(3-hydroxy-phenyl)-methanone; (3H-benzo[e]indol-2-yl)-(2-chloro-pyridin-4-yl)-methanone; (3H-benzo[e]indol-2-yl)-(1-oxy-pyridin-4-yl)-methanone; Phenyl-(6,7,8,9-tetrahydro-3H-benzo[e]indol-2-yl)-methanone; (3H-Benzo[e]indol-2-yl)-(4-hydroxy-3-methoxylthenyl)-methanone; (3H-Benzo[e]indol-2-yl)-(4-benzyloxy-3-methoxy-phenyl)-methanone; 4-(3H-Benzo[e]-indole-2-carbonyl)-benzoic acid methyl ester; 4-(3H-Benzo[e]indole-2-carbonyl)-benzoic acid; (4-Amino-phenyl)-(3H-benzo[e]indol-2-yl)-methanone; 5-(3H-Benzo[e]indole-2-carbonyl)-2-benzyloxy-benzoic acid methyl; 5-(3H-Benzo[e]indole-2-carbonyl)-2-benzyloxy-benzoic Acidmethanone; (3H-Benzo[e]indol-2-yl)-(2-methoxy-pyridin-4-yl)-methanone; (5-Fluoro-3H-benzo[e]indol-2-yl)-(3-methoxy-phenyl)-methanone; (5-Fluoro-3H-benzo[e]indol-2-yl)-pyridin-4-yl-methanone; (4-Benzyloxy-3-methoxy-phenyl)-(5-fluoro-3H-benzo[e]indol-2-yl)-methanone; (5-Fluoro-3H-benzo[e]indol-2-yl)-(4-hydroxy-3-methoxy-phenyl)-methanone; (3H-Benzo[e]indol-2-yl)-(3-hydroxy-methyl-phenyl)-methanone; Cyclohexyl-(5-fluoro-3H-benzo[e]indol-2-yl)-methanone; (5-Fluoro-3H-benzo[e]indol-2-yl)-(3-fluoro-4-hydroxy-phenyl)-methanone; (3H-Benzo[e]indol-2-yl)-p-tolyl-methanone; (3H-Benzo[e]indol-2-yl)-(3-methoxy-phenyl-methanol; (3H-Benzo[e]indol-2-yl)-pyridin-4-yl-methanol; 3H-Benzo[e]indole-2-carbox-ylic acid phenylamide; 3H-Benzo[e]indole-2-carboxylic acid (3-methoxy-phenyl)-amide; (3H-Benzo[e]indol-2-yl)-(4-dimethylamino-phenyl)-methanone; (4-Amino-3-methoxy-phen-yl)-(3H-benzo[e]indol-2-yl)-methanone; (4-Amino-3-methoxy-phenyl)-(5-hydroxy-3H-benzo[e]indol-2-yl)-methanone; (4-Amino-3-methoxy-phenyl)-(5-methoxy-3H-benzo[e]-indol-2-yl)-methanone; N-[4-(3H-Benzo[e]indole-2-carbonyl)-phenyl]-methane-sulfonamide; 3H-Benzo[e]indole-2-carboxylic acid (4-amino-phenyl)-amide; (4-Amino-phen-yl)-(5-methoxy-3H-benzo[e]indol-2-yl)-methanone; (4-Amino-2-fluoro-phenyl)-(5-meth-oxy-3H-benzo[e]indol-2-yl)-methanone; (4-Amino-3-fluoro-phenyl)-(5-methoxy-3H-benzo[e]indol-2-yl)-methanone; (4-Amino-2-methoxy-phenyl)-(5-methoxy-3H-benzo[e]-indol-2-yl)-methanone; (4-Amino-phenyl)-(9-methoxy-3H-benzo[e]indol-2-yl)-methanone; (4-Amino-3-methoxy-phenyl)-(9-methoxy-3H-benzo[e]indol-2-yl)-methanone; (4-Amino-2-methoxy-phenyl)-(9-methoxy-3H-benzo[e]indol-2-yl)-methanone; (4-Amino-3-fluoro-phenyl)-(9-methoxy-3H-benzo[e]indol-2-yl)-methanone; (4-Amino-2-fluoro-phen-yl)-(9-methoxy-3H-benzo[e]indol-2-yl)-methanone; (4-Amino-3-fluoro-phenyl)-(3H-benzo[e]indol-2-yl)-methanone; (4-Amino-2-fluoro-phenyl)-(3H-benzo[e]indol-2-yl)-methanone; (4-Amino-phenyl)-(7-methoxy-3H-benzo[e]indol-2-yl)-methanone; (4-Amino-phenyl)-(5-hydroxy-3-methyl-3H-benzo[e]indol-2-yl)-methanone; (7-Amino-5-fluoro-9-hydroxy-3H-benzo[e]indol-2-yl)-(3-methyl-pyridin-4-yl)-methanone; (5-Amino-3H-pyrrolo[3,2-f]isoquinolin-2-yl)-(3-methoxy-pyridin-4-yl)-methanone; (4-Amino-2-methyl-phenyl)-(9-hydroxy-3H-pyrrolo[2,3-c]quinolin-2-yl)-methanone; and (4-Amino-phenyl)-(7-methanesulfonyl-3H-benzo[e]indol-2-yl)-methanone, or a salt thereof.

In some embodiments, the PFKFB3 inhibitor administered according to the provided methods is at least one of: 1-Pyridin-4-yl-3-quinolin-4-yl-propenone; 1-Pyridin-4-yl-3-quinolin-3-yl-propenone; 1-Pyridin-3-yl-3-quinolin-2-yl-propenone; 1-Pyridin-3-yl-3-quinolin-4-yl-propenone; l-Pyridin-3-yl-3-quinolin-3-yl-propenone; l-Naphthalen-2-yl-3-quinolin-2-yl-propenone; l-Naphthalen-2-yl-3-quinolin-3-yl-propenone; l-Pyridin-4-yl-3-quinolin-3-yl-propenone; 3-(4-Hydroxy-quinolin-2-yl)-1-pyridin-4-yl-propenone; 3-(8-Hydroxy-quinolin-2-yl)-1-pyridin-3-yl-propenone; 3-Quinolin-2-yl-1-p-tolyl-propenone; 3-(8-Hydroxy-quinolin-2-yl)-1-pyridin-4-yl-propenone; 3-(8-Hydroxy-quinolin-2-yl)-1-p-tolyl-propenone; 3-(4-Hydroxy-quinolin-2-yl)-1-p-tolyl-propenone; 1-Phenyl-3-quin-olin-2-yl-propenone; 1-Pyridin-2-yl-3-quinolin-2-yl-propenone; 1-(2-Hydroxy-phenyl)-3-quinolin-2-yl-propenone; 1-(4-Hydroxy-phenyl)-3-quinolin-2-yl-propenone; 1-(2-Amino-phenyl)-3-quinolin-2-yl-propenone; 1-(4-Amino-phenyl)-3-quinolin-2-yl-propenone; or a salt thereof.

In some embodiments, the PFKFB3 inhibitor administered according to the provided methods is at least one of: 4-(3-Quinolin-2-yl-acryloyl)-benzamide; 4-(3-Quinolin-2-yl-acryloyl)-benzoic acid; 3-(8-Methyl-quinolin-2-yl)-1-pyridin-4-yl-propenone; 1-(2-Fluoro-pyridin-4-yl)-3-quinolin-2-yl-propenone; 3-(8-Fluoro-quinolin-2-yl)-1-pyridin-4-yl-propenone; 3-(6-Hydroxy-quinolin-2-yl)-1-pyridin-4-yl-propenone; 3-(8-Methylamino-quinolin-2-yl)-1-pyridin-4-yl-propenone; 3-(7-Methyl-quinolin-2-yl)-1-pyridin-4-yl-propenone; and 1-Methyl-4-[3-(8-methyl-quinolin-2-yl)-acryloyl]-pyridinium, or a salt thereof.

In some embodiments, the PFKFB3 inhibitor administered according to the provided methods is PFK15 (1-(4-pyridinyl)-3-(2-quinolinyl)-2-propen-1-one); (2S)—N-[4-[[3-Cyano-1-(2-methyl-propyl)-1H-indol-5-yl]oxy]phenyl]-2-pyrrolidine-carboxamide 3PO (3-(3-Pyridinyl)-1-(4-pyridinyl)-2-propen-1-one); (2S)—N-[4-[[3-Cyano-1-[(3,5-dimethyl-4-isoxazolyl)methyl]-1H-indol-5-yl]oxy]phenyl]-2-pyrrolidine-carboxamide; and Ethyl 7-hydroxy-2-oxo-2H-1-benzopyran-3-carboxylate, or a salt thereof.

In a particular embodiment, the PFKFB3 inhibitor administered according to the provided methods is PFK15, or a salt thereof.

In a particular embodiment, the PFKFB3 inhibitor administered according to the provided methods is PFK158 ((E)-1-(4-Pyridinyl)-3-[7-(trifluoromethyl)-2-quinolinyl]-2-propen-1-one), or a salt thereof.

In a particular embodiment, the PFKFB3 inhibitor administered according to the provided methods is BrAcNHEtOP (N-bromoacetylethanolamine phosphate), or a salt thereof.

In a particular embodiment, the PFKFB3 inhibitor administered according to the provided methods is AZ67, or a salt thereof.

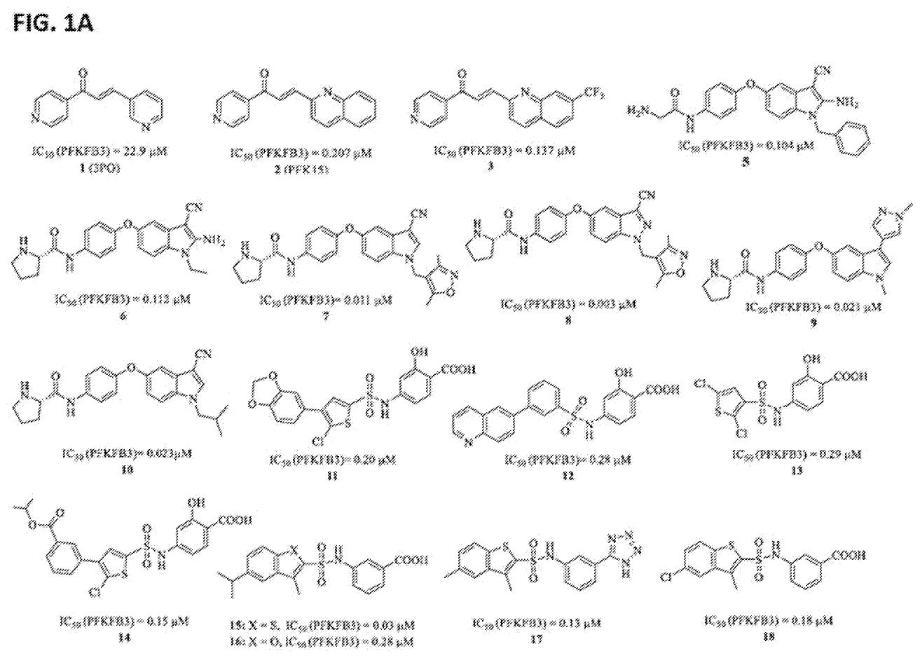

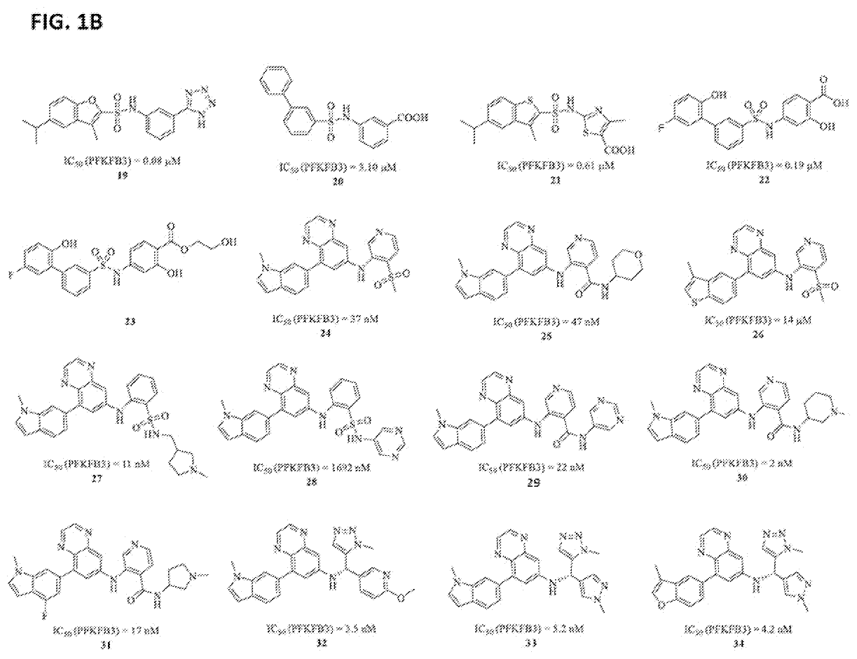

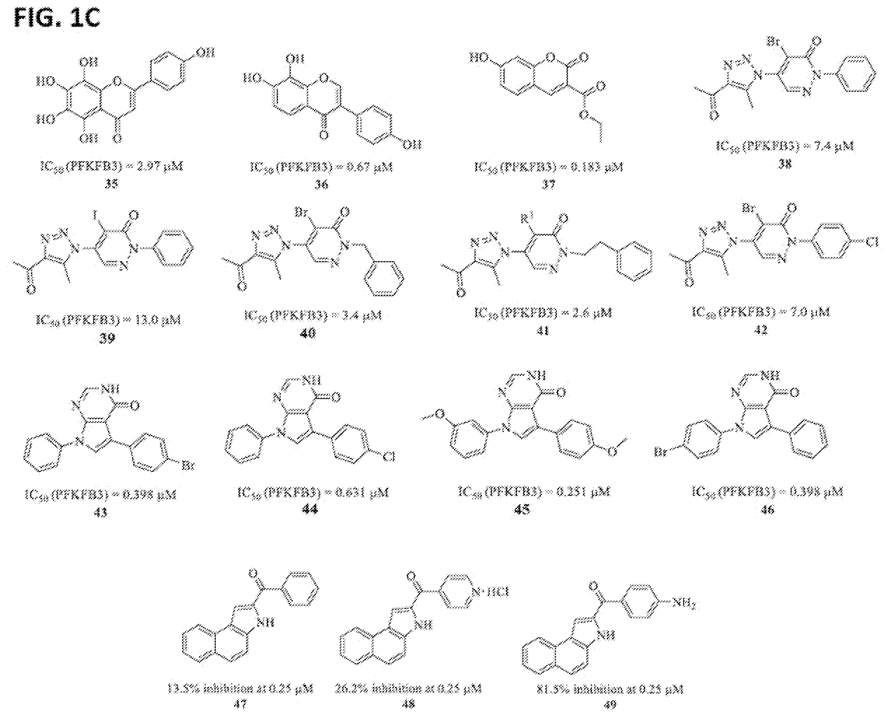

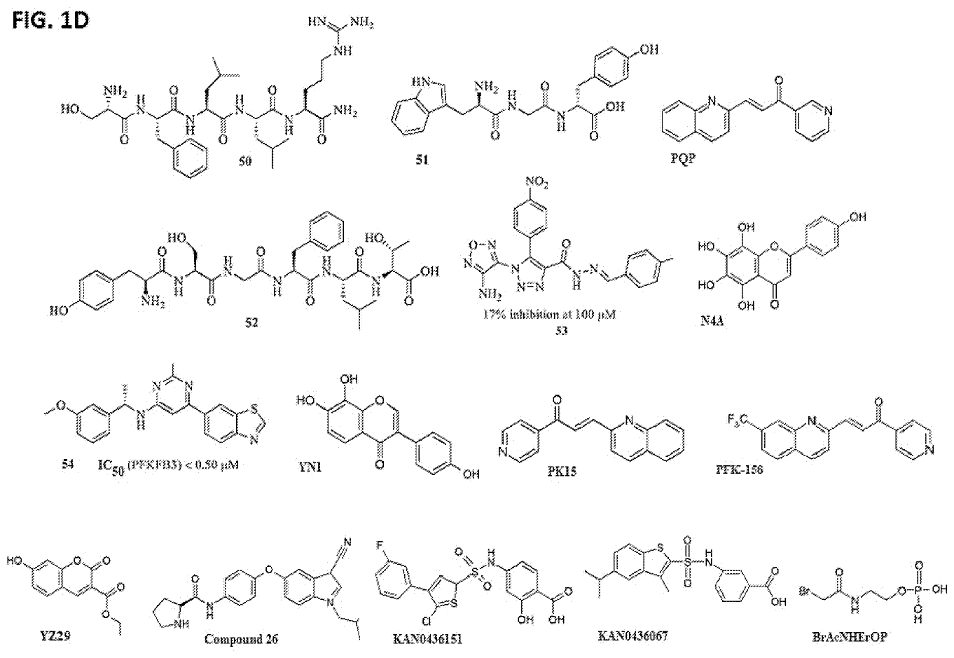

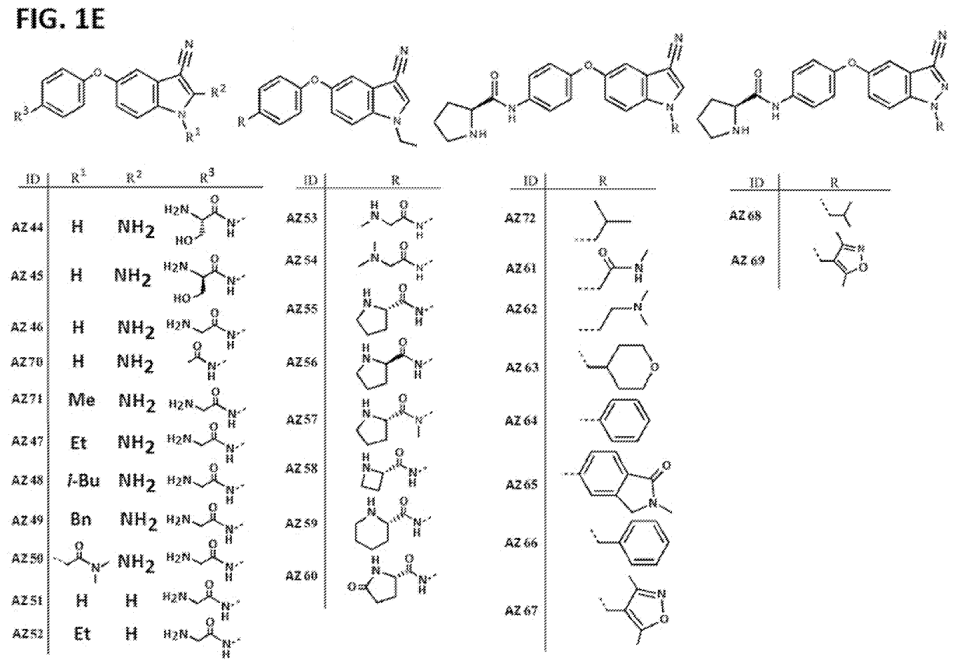

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In a particular embodiment, the PFKFB3 inhibitor administered according to the provided methods is KAN0436151, or a salt thereof.

In a particular embodiment, the PFKFB3 inhibitor administered according to the provided methods is KAN0436067, or a salt thereof.

### HIF1-α Pathway Inhibitors

Hypoxia-inducible factor 1-alpha (HIF-1-alpha) is a subunit of a heterodimeric transcription factor hypoxia-inducible factor 1 (HIF-1) that is considered to be the master transcriptional regulator of cellular and developmental response to hypoxia.

In some embodiments, the disclosure provides a method of treating a neurological condition in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor; and
wherein the PFKFB3 inhibitor does not inhibit PI3K/AKT/mTOR pathway or HIF1-α.

In further embodiments, the neurological condition treated according to a method provided herein is a neurodegenerative disease such as Alzheimer's (AD), Parkinson's (PD), Huntington's diseases (HD), amyotrophic lateral sclerosis (ALS), and multiple sclerosis (MS), or neural trauma.

The term “HIF1-α Pathway-α Inhibitor” as used herein refers to a composition that inhibits or reduces HIF1-α directly or indirectly via inhibiting one or more activities of the PI3K/AKT/mTOR pathway that is upstream of the HIF1-α pathway. The term “HIF1-α Inhibitor” is used herein to refer to a composition that inhibits or reduces HIF1-α directly. Thus, for example, mTOR pathway inhibitors such as temsirolimus, everolimus, and sirolimus are considered herein to be “HIF1-α Pathway-α Inhibitors”, but not “HIF1-α Inhibitors.”

The “HIF1-α Pathway-α Inhibitors” that can be administered according to the provided methods are not particularly limited. In some embodiments, the administered HIF1-α Pathway Inhibitor is an antibody or a HIF1-α-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor

In some embodiments, the administered HIF1-α Pathway Inhibitor administered according to the provided methods has an IC50 for a HIF1-α activity/function of 100 μM or lower concentration for a HIF1-α activity. In some embodiments, the HIF1-α Pathway Inhibitor has an IC50 of at least or at most or about 200, 100, 80, 50, 40, 20, 10, 5, or 1 M, or at least or at most or about 100, 10, or 1 nM, or lower (or any range or value derivable therefrom). In some embodiments, the HIF1-α Pathway Inhibitor inhibits the expression of HIF1-α. Assays for determining the ability of a compound to inhibit HIF1-α activity are known in the art. In some embodiments, the inhibition of HIF1-α activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit HIF1-α activity.

The HIF1-α Inhibitors that can be administered according to the provided methods are not particularly limited. In some embodiments, the HIF1-α Inhibitor modulates one or more of HIF-1α mRNA expression; HIF-1α protein translation or degradation; HIF-1α/HIF-10 dimerization; HIF-1α-DNA binding (e.g., HIF-1α/HRE); and/or HIF-1α transcriptional activity (e.g., CH-1 of p300/C-TAD of HIF-1α).

In some embodiments, the HIF1-α Inhibitor administered according to the provided methods is a small molecule. In some embodiments, the HIF1-α Inhibitor administered according to the provided methods is a protein or polypeptide (e.g., an anti HIF1 antibody or antibody fragment that binds HIF1). In some embodiments, the HIF1-α Inhibitor administered according to the provided methods is a therapeutic nucleic acid (e.g., an aptamer, antisense molecule, ribozyme, a Dicer substrate, siRNA, miRNA, dsRNA, ssRNA, or a shRNA).

In some embodiments, the HIF1-α Pathway Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor); a HIF translation inhibitor (e.g., a topoisomerase inhibitor, a microtubule targeting drug a cardiac glycoside, or an antisense HIF-1α mRNA); an inhibitor of HIF stability, nuclear localization or dimerization (e.g., acriflavine or an HDAC inhibitor); an inhibitor of HIF transactivation (e.g., a HIF1 coactivator recruitment inhibitor or a HIF1 DNA binding inhibitor).

In some embodiments, the HIF1-α Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor). In some embodiments, the HIF1-α Inhibitor administered according to the provided methods is a PI3K pathway inhibitor. In one embodiment, the administered HIF1-α Pathway Inhibitor is P3155, LY29, LY294002, wortmannin, or GDC-0941. In one embodiment, the administered HIF1-α Pathway Inhibitor is resveratrol. In another embodiment, the administered HIF1-α Pathway Inhibitor is a glyceolin. In some embodiments, the HIF1-α Pathway Inhibitor administered according to the provided methods is an mTOR inhibitor. In one embodiment, the administered HIF1-α Pathway Inhibitor is rapamycin, temsirolimus (CC1-779), everolimus, sirolimus, or PP242.

In a particular embodiment, the administered HIF1-α Inhibitor is silibinin.

In some embodiments, the HIF1-α Inhibitor administered according to the provided methods is a HIF translation inhibitor. In one embodiment, the administered HIF1-α Inhibitor is PX-478 (S-2-amino-3-[4′-N,N-bis(chloroethyl)[amino]phenyl propionic acid N-oxide dihydrochloride), NSC-64421, camptothecin (CPT), SN38, irinotecan, topotecan, NSC-644221, cycloheximide, or apigenin, or a salt thereof. In one embodiment, the administered HIF1-α Inhibitor is aminoflavone, KC7F2 (N,N′-(disulfanediylbis(ethane-2,1-diyl))bis(2,5-dichlorobenzene-sulfonamide), 2-meth-oxyestra-diol (2ME2) or an analog or salt thereof. In one embodiment, the administered HIF1-α Inhibitor is ENMD-1198, ENMD-1200, or ENMD-1237, or a salt thereof. In one embodiment, the administered HIF1-α Inhibitor is EZN-2208, or a salt thereof.

In a particular embodiment, the administered HIF1-α Inhibitor is PX-478, or a salt thereof.

In some embodiments, the HIF1-α Inhibitor administered according to the provided methods is a cardiac glycoside. In one embodiment, the administered cardiac glycoside is digoxin, or a salt thereof. In another embodiment, the administered cardiac glycoside ouabain or proscillardin A, or a salt thereof.

In some embodiments, the HIF1-α Pathway Inhibitor administered according to the provided methods is a topoisomerase inhibitor. In one embodiment, the administered topoisomerase inhibitor is camptothecin (CPT), SN38, irinotecan, or topotecan (e.g., PEG-SN38), or a salt thereof.

In some embodiments, the HIF1-α Pathway Inhibitor administered according to the provided methods is a microtubule targeting drug. In one embodiment, the administered microtubule targeting drug is 2 methoxyestradiol (2ME2), ENMD-1198, ENMD-1200, ENMD-1237, or Taxotere, or a salt thereof.

In some embodiments, the HIF1-α Inhibitor administered according to the provided methods is a therapeutic nucleic acid. In some embodiments therapeutic nucleic acid is an aptamer, antisense molecule, ribozyme, a Dicer substrate, siRNA, miRNA, dsRNA, ssRNA, and shRNA). In some embodiments, therapeutic nucleic acid is an antisense oligonucleotide.

In some embodiments, the HIF1-A Inhibitor administered according to the provided methods is a siRNA or an antisense oligonucleotide. In one embodiment, the administered HIF1-α Inhibitor is EZN-2968. In one embodiment, the administered HIF1-α Inhibitor is RX-0047.

Representative examples of human HIF1-A coding sequences are provided in GenBank accession numbers NM_004566.3, NM_001145443.2, NP_001138915.1, NM_001282630.2, NM_001314063.1. NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory (therapeutic) nucleic acid include but are not limited to, antisense nucleic acids such as: ENMD-1198 (small interfering RNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%, 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is an inhibitor of HIF stability, nuclear localization or dimerization. In one embodiment, the inhibitor administered according to the provided methods destabilizes HIF. In one embodiment, the inhibitor administered according to the provided methods is a histone deacetylase inhibitor (HDACI). In a further embodiment, the administered HDACI is LW6/CAY10585, vorinostat, romidepsin (FK228), panobinostat, belinostat, Trichostatin A (TSA), LAQ824, or phenethyl isothiocyanate, or a salt thereof. In one embodiment, the inhibitor administered according to the provided methods is PX-12/pleurotin, HIF-1α inhibitor (CAS No. 934593-90-5), cryptotanshinone, or BAY 87-2243 (1-cyclopropyl-4-[4-[[5-methyl-3-[3-[4-(trifluoromethoxy) phenyl]-1,2,4-oxadiazol-5-yl]-1H-pyrazol-1-yl]methyl]-2-pyridinyl]-piperazine), or a salt thereof. In one embodiment, the inhibitor administered according to the provided methods is IDF-11774, Bisphenol A/Dimethyl bisphenol A, or a salt thereof. Chrysin (5,7-dihydroxy-flavone), or SCH66336, or a salt thereof. In one embodiment, the inhibitor administered according to the provided methods is geldanamycin or analog thereof, 17-AAG (tanespimycin: allylamino-17-demethoxygeldanamycin), 17-DMAG (alvespimycin), 17AG, radiccicol, KF58333, ENMD-1198, ENMD-1237, or ganetasipib (ST-9090), or a salt thereof. In one embodiment, the inhibitor administered according to the provided methods interferes with HIF-dimerization. In one embodiment, the inhibitor administered according to the provided methods is acriflavine, or a salt thereof. In one embodiment, the inhibitor administered according to the provided methods is TC-S7009, PT2385, or TAT-cyclo-CLLFVY, or a salt thereof.

In a particular embodiment, the inhibitor administered according to the provided methods is ganetasipib, or a salt thereof.

In a particular embodiment, the inhibitor administered according to the provided methods is BAY 87-2243.

In some embodiments, the HIF1-α Pathway Inhibitor administered according to the provided methods is a histone deacetylase inhibitor (HDACI). In one embodiment, the administered HDACI is LW6/CAY10585 (methyl 3-(2-(4-(adamantan-1-yl)phenoxy)acetamido)-4-hydroxy-benzoate, vorinostat, romidepsin (FK228), panobinostat, belinostat, Trichostatin A (TSA), LAQ824, or phenethyl isothiocyanate, or a salt thereof.

In some embodiments, the HIF1-α Pathway Inhibitor administered according to the provided methods is a heat shock protein inhibitor. In one embodiment, the administered HIF1-α Pathway Inhibitor is an HSP90 inhibitor. In one embodiment, the administered HSP90 inhibitor is a geldanamycin or analog thereof, 17-AAG (tanespimycin: allylamino-17-demethoxy geldanamycin), 17-DMAG (alvespimycin), 17AG, radiccicol, KF58333, ENMD-1198, ENMD-1237, or ganetasipib, or a salt thereof. In a particular embodiment, the administered heat shock protein inhibitor is ganetasipib, or a salt thereof. In one embodiment, the administered HIF1-α Pathway Inhibitor is an HSP70 inhibitor. In one embodiment, the administered HSP70 inhibitor is triptolide, or a salt thereof.

In some embodiments, the HIF1-α Inhibitor administered according to the provided methods is an inhibitor of HIF transactivation. In one embodiment, the HIF1-α Inhibitor administered according to the provided methods inhibits HIF coactivator recruitment. In one embodiment, the administered HIF1-α Inhibitor is chetomin, YC-1, or KCN-1 (3,4-dimethoxy-N-[(2,2-dimethyl-2H-chromen-6-yl)methyl]-N-phenylbenzenesulfonamide), or a salt thereof. In another particular embodiment, the administered HIF1-α Inhibitor is NSC 607097, or a salt thereof. In one embodiment, the administered HIF1-α Inhibitor is a proteasome inhibitor. In a further embodiment, the administered inhibitor is bortezomib or carfilzomib, or a salt thereof. In one embodiment, the administered HIF1-α Inhibitor is indenopyrazole 21, FM19G11, flavopiridol, Amphotericin B, actinomycin, AJM290, or AW464, or a salt thereof. In one embodiment, the administered HIF1-α Inhibitor is triptolide, or a salt thereof.

In a particular embodiment, the HIF1-α Inhibitor administered according to the provided methods is YC-1, or a salt thereof.

In some embodiments, the HIF1-α Inhibitor administered according to the provided methods is an antibody that binds HIF1-α or a HIF1-α-binding antibody fragment (e.g., a single chain antibody, a single-domain antibody (e.g., the AG1-5 VHH), a Fab fragment, F(ab′)2 fragment, Fd fragment; Fv fragment, scFv, dAb fragment, or another engineered molecule, such as a diabody, triabody, tetrabody, minibody, and a minimal recognition unit). In a particular embodiment, the administered HIF1-α Inhibitor is a VHH or nanobody. In one embodiment, the administered antibody is AGI-5. In one embodiment, the administered antibody is AHPC.

In some embodiments, the HIF1-α Inhibitor administered according to the provided methods is an inhibitor of HIF1 DNA-binding. In one embodiment, the administered HIF1-α Inhibitor is echinomycin (NSC-13502) or Compound DJ12.162. In one embodiment, the administered HIF1-α Inhibitor is an anthracycline. In a further embodiment, the administered inhibitor is doxorubicin or danuorubicin. In one embodiment, the administered HIF1-α Inhibitor is a polyamide. In some embodiments, the HIF1-α Inhibitor is an antibody that binds HIF1-α or is a HIF1-α-binding antibody fragment such as a VHH or nanobody.

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, MiRNA, dsRNA, ssRNA, and shRNA). In some embodiments, the therapeutic nucleic acid is ENMD-1198 or an antisense oligonucleotide.

In some embodiments, the HIF1-A Inhibitor administered according to the provided methods is an siRNA or an antisense oligonucleotide. In some embodiments, the administered HIF1-A Inhibitor is ENMD-1198. In some embodiments, the administered HIF1-A Inhibitor is EZN-2968.

Representative examples of human HIF1-A coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory nucleic acid include but are not limited to, antisense nucleic acids such as: ENMD-1198 (small interfering RNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%, 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is a therapeutic nucleic acid. In some embodiments the therapeutic nucleic acid is an ENMD-1198 molecule or antisense oligonucleotide.

### Kits for Administration of Active Agents

In another embodiment, the disclosure provides a kit containing a HIF1-α Pathway Inhibitor and a PFKFB3 inhibitor and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the lipid is in one vial, and the therapeutic agent is in a separate vial. The kit may include, for example, at least one inhibitor of PFKFB3 expression/activity, at least one inhibitor of HIF1-alpha expression/activity, and one or more reagents to prepare, formulate, and/or administer the components described herein or perform one or more steps of the methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. For example, the kit may comprise instructions for use of the HIF1-α Pathway Inhibitor and a PFKFB3 inhibitor and/or other therapeutic for the treatment of a neurological condition such as a neurodegenerative disease or neurotrauma in a subject. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression of PFKFB3 and/or HIF-α or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling PFKFB3 and/or HIF-1 alpha in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. Alternatively, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained. The kit may also include instructions for employing the kit components as well the use of any other reagent not included in the kit. Instructions may include variations that can be implemented.

### Methods of Administration

The regimen of administration according to a method provided herein (e.g., dose combined with frequency of administration) will generally involve administration in an amount and at a frequency to provide for a desired effect, e.g., administration of an amount effective to provide for improvement in one or more symptoms of a neurological condition in a subject such as one or more symptoms associated with a neurodegenerative disease such as PD or AD, or neurotrauma. Administration of the compositions will typically be via any common route. This includes, but is not limited to oral, parenteral, orthotopic, intradermal, subcutaneous, intramuscular, intraperitoneal, intranasal, intratumoral, or intravenous injection. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, or about 25% to about 70%. Typically, compositions are administered in a manner compatible with the dosage formulation, and in such amount as will be therapeutically effective. The quantity to be administered depends on the subject to be treated. Precise amounts of active ingredient required to be administered depend on the judgment of the practitioner.

In some embodiments, the compositions are administered parenterally. The phrases “parenteral administration” and “administered parenterally” as used herein refer to modes of administration other than enteral and topical administration, such as injections, and include without limitation intravenous, intramuscular, intrapleural, intravascular, intrapericardial intraarterial, intrathecal, intracapsular, intraorbital, intracardiac, intradrenal, intraperitoneal, transtracheal, subcutaneous, subcuticular, intra-articular, subcapsular, subarachnoid, intraspinal and intrasternal injection and infusion.

In particular embodiments, the provided compositions are administered such that they come into contact with neural cells or neural tissue, such as central nervous system (CNS) cells or tissue. Such tissue includes brain and spinal cord (e.g., cervical, thoracic, or lumbar) tissue. As such, in some embodiments compositions provided herein are administered to treat neural cells/tissue in vivo via direct intracranial injection or injection into the cerebrospinal fluid. Alternatively, the provided compounds can be administered systemically (e.g. intravenously) and may come into contact with the affected neural tissue through other mechanisms such as lesions (where the blood-brain barrier is compromised). In some embodiments, the administered compositions are in a form capable of crossing the blood-brain barrier and entering the neural system (e.g., CNS). In further embodiments, the administered compositions are formulated for such administration to neural tissue.

Formulations for parenteral administration are conveniently sterile aqueous preparations of the active agent, which preparations are preferably isotonic with the blood of the intended recipient. These preparations can be administered by means of subcutaneous, intravenous, intramuscular, or intradermal injection. Such preparations can conveniently be prepared by admixing the compound with water or a glycine buffer and rendering the resulting solution sterile and isotonic with the blood.

In some embodiments, the compositions are administered orally. Formulations suitable for oral administration can be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations can be prepared by any suitable method of pharmacy which includes the step of bringing into association the active compound and a suitable carrier (which may contain one or more accessory ingredients as noted above). In general, the provided formulations are prepared by uniformly and intimately admixing the active compound with a liquid or finely divided solid carrier, or both, and then, if necessary, shaping the resulting mixture. For example, a tablet can be prepared by compressing or molding a powder or granule containing the active agent, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

Formulations suitable for buccal (sub-lingual) administration include lozenges having the active agent in a flavored base, usually sucrose and acacia or tragacanth; and pastilles containing the active agent in an inert base such as gelatin and glycerin or sucrose and acacia.

Formulations suitable for topical application (e.g., in the oral passage, nasopharynx, or oropharynx) take the form of an ointment, cream, lotion, paste, gel, spray, aerosol, or oil. Carriers which can be used include vaseline, lanoline, polyethylene glycols, alcohols, transdermal enhancers, and combinations of two or more thereof.

In some embodiment, the disclosure provides a method of treatment wherein the compositions provided herein are administered in combination with one or more additional Therapeutic agent(s) (e.g., Therapeutic agents used to prevent and/or treat neural diseases and/or conditions or their associated effects (e.g., pain)). The combination of the provided compositions and Therapeutic agent(s) may be administered or co-administered (e.g., consecutively, simultaneously, at different times) in any conventional dosage form. Co-administration in the present context refers to the administration of more than one Therapeutic agent to a subject in the course of a coordinated treatment to achieve an improved clinical outcome. Such co-administration may also be coextensive, that is, occurring during overlapping periods of time. For example, a first Therapeutic agent may be administered to a patient before, concomitantly, before and after, or after a second active agent is administered. In some embodiments the Therapeutic agents are combined/formulated in a single composition and thus administered to the subject at the same time.

### Methods of Treatment and Use

### Neurological Conditions

The provided methods and compositions have uses in treating neurological conditions. In one embodiment, the disclosure provides a method of treating a neurological condition in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the neurological condition treated according to the provided methods is an injury resulting from, or a condition associated with: mild cognitive impairment (MCI); aging-related memory impairment (AAMI); neuropathy; a neurodegenerative disease; a seizure-related injury; multiple sclerosis; amyotrophic lateral sclerosis; a stroke-related injury; a cerebral aneurism-related injury; a spinal cord injury (e.g. a contusion, compression, or laceration); a concussion-related injury (including post-concussion syndrome); cerebral ischemia; or traumatic brain injury.

In some embodiments, the neurological condition treated according to the provided methods is mild cognitive impairment (MCI) or aging-related memory impairment (AAMI).

In some embodiments, the neurological condition treated according to the provided methods is a neuropathy.

In some embodiments, the neurological condition treated according to the provided methods is a neurodegenerative disease. In some embodiments, the treated neurodegenerative disease is Alzheimer's disease (AD), Parkinson's disease (PD), or Huntington's disease (HD). In particular embodiments, the treated neurodegenerative disease is AD. In particular embodiments, the treated neurodegenerative disease is PD. In particular embodiments, the treated neurodegenerative disease is HD.

In some embodiments, the neurodegenerative disease treated according to the provided methods is selected from: amyotrophic lateral sclerosis (ALS), Friedreich's ataxia, and frontotemporal lobar degeneration.

In some embodiments, the neurological condition treated according to the provided methods is dementia (e.g., AD-type senile dementia (SDAT), vascular dementia, or dementia with Lewy bodies).

In some embodiments, the neurological condition treated according to the provided methods is neural trauma. In some embodiments, the neurotrauma is injury to the central nervous system (CNS). In one embodiment, the injury to the CNS is spinal cord injury (SCI). In a further embodiment, the SCI is acute SCI. In one embodiment, the injury to the CNS is traumatic brain injury. In one embodiment, the injury to the CNS treated according to the provided methods is cerebral ischemia.

In some embodiments, the subject treated according to the provided methods is at risk of having an neurological condition. In some embodiments, the provided methods are performed as a prophylactic treatment for a neurological condition.

In some embodiments, the provided methods and compositions prevent a neurological condition in a subject at risk for developing the neurological condition, e.g., a subject having one or more risk factors associated with development of the neurological condition. In some embodiments, the subject has one or more risk factors selected from: over 55 years of age, smoking, obesity, diabetes, metabolic syndrome, heart disease, high blood pressure, stroke, myocardial infarction, family history of a neurological condition, exposure to solvents, exposure to pesticides, and head injury or brain.

In some embodiments, the subject treated according to the provided methods has, or has been diagnosed as having, a neurological condition. The development of neurological conditions such as neurodegenerative disease can routinely be detected and assessed using standard clinical techniques known in the art, such as functional tests including cognitive tests, behavioral reflex tests; balance, coordination and gait tests, and deep tendon reflex tests); qualitative examinations of mental status, cranial nerves, motor system, sensory system; biochemical assays; nerve and muscle biopsy; and computerized axial tomography (CAT) scan, computerized tomography (CT), nuclear magnetic resonance (MRI), Transcranial Doppler, neurosonography, electroencephalogram (EEG), SPECT scan, positive electricity Sub-emission computed tomography art (PET), or diffusion-weighted imaging (DWI).

In some embodiments, the disclosure provides methods and compositions that prevent, inhibit or delay the onset of an neurological condition by administering compositions provided herein to a subject before the onset of the neurological condition, e.g., before the onset of one or more symptoms thereof. In some embodiments, the HIF1-α Pathway Inhibitor and the PFKFB3 inhibitor are administered before the onset of one or more symptoms of the neurological condition. In some embodiments, treating a neurological condition according to a method provided herein comprises delaying the onset of one or more symptoms of a neurological condition.

In some embodiments, the HIF1-α Pathway Inhibitor and the PFKFB3 inhibitor are administered after the onset of one or more symptoms of a neurological condition. In some embodiments, the HIF1-α Pathway Inhibitor and the PFKFB3 inhibitor are administered to inhibit the progression of a neurological condition. In some embodiments, the provided methods and compositions are used to treat one or more different stages of the neurological condition.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating neurological condition is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating a neurological condition according to a method provided herein comprises reducing one or more symptoms of the neurological condition in the subject compared to the subject prior to treatment with the HIF1-α Pathway Inhibitor and the PFKFB3 inhibitor. In some embodiments, the one or more reduced symptoms of the neurological condition is selected from: memory loss, cognitive impairment, personality changes, depression, mood swings, unsteady gait, twitching and jerking movements and tremors, dementia, slurred speech, drooling, impaired judgement, difficulty in swallowing, rigid muscles, impaired posture and balance; loss of automatic movements, difficulty writing, reduction in apoptosis/destruction/loss of the number and/or function of neural cells and/or tissue; increase survival of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; and increased survival/survival time. In some embodiments, the one or more symptoms of neurological condition are reduced by at least 10%, at least 20%, at least 30%, at least 40%, or at least 50% compared to the subject prior to treatment with the HIF1-α Pathway Inhibitor and the PFKFB3 inhibitor.

Neurological tests for identifying and monitoring a neurological condition are known in the art. Diagnosis, treatment, prevention, and progression of an neurological condition can routinely be assessed using a variety of techniques known in the art. In some embodiments, treatment of the neurological condition comprises reducing or eliminating one or more of the above symptoms. In some embodiments, treatment comprises reducing or eliminating one or more of apoptosis, inflammation, and/or ischemic reperfusion injury or neural cells or neural tissue in a subject in need thereof. In some embodiments, treatment comprises reducing or eliminating a cognitive, motor and/or sensory impairment in a subject in need thereof.

The term “neurological test,” as used herein, refers to any test, presently known or unknown, that is useful for ascertaining and/or measuring neurological activity and encompasses anatomically-based testing, functional testing, and biochemical assays. Exemplary neurological tests include, without limitation, CAT scan, nuclear magnetic resonance (MRI), Transcranial Doppler, neurosonography, electroencephalogram (EEG), SPECT scan, computerized tomography (CT) scanning, positive electricity Sub-emission computed tomography art (PET), diffusion-weighted imaging (DWI), as well as more qualitative examinations of mental status, cranial nerves, motor system, sensory system, the deep tendon reflexes, coordination, and gait.

In some embodiments, treating a neurological condition according to a method provided herein reduces one or more cognitive, behavioral and/or physical impairments in the subject. In some embodiments, treating a neurological condition according to a method provided herein comprises increasing or improving one or more neurological parameters in the subject compared in the subject prior to treatment with the HIF1-α Pathway Inhibitor and the PFKFB3 inhibitor. In some embodiments, the one or more improved neurological parameters are selected from: impaired cognition and/or memory; impaired balance, coordination and/or gait; impaired reflexes; tremors, twitching and/or jerking movements; mood swings; dementia; impaired speech and/or writing; and loss of neuronal cells. Each of these neurological parameters can routinely be monitored using techniques described herein or otherwise known in the art.

In some embodiments, the administration of the provided compositions have use in (i) improving cognition and/or (ii) treating and/or preventing cognitive impairment. In some embodiments, the administration of the provided compositions have use in (i) improving cognition and/or (ii) treating and/or preventing cognitive dysfunction in a subject. The cognitive function in human subjects can routinely be determined using methods known in the art including for example, the RAVLT cognitive test, Repeatable Battery for the Assessment of Neuropsychological Status, or California Verbal Learning Test). In some embodiments, the cognition of the subject is assessed using a Wechsler memory scale [WMS]-verbal paired associates (VPA) test; a Rey Auditory Verbal Learning test recall [RAVLT recall], a Recognition test, a WMS digit span test, a Controlled Word Association test [COWAT], a category fluency test, a Trail Making Test [TMT], an orientation task ADAS-cog test, or a letter digit substitution test.

In some embodiments, the provided methods prevent, reduce or delay the neurological condition. In some embodiments, the provided methods are administered to a subject at risk for developing a neurological condition. In such subjects, prevention of a neurological condition may be monitored by the absence of typical hallmarks of the neurological condition. For example, subjects to whom an effective amount of a HIF1-alpha inhibitor and PFKFB3 inhibitor is administered prophylactically may not experience or may experience a reduced incidence of one or more of the following symptoms: impaired cognition and/or memory; impaired balance, coordination and/or gait; impaired reflexes; tremors, twitching and/or jerking movements; mood swings; dementia; impaired speech and/or writing; and loss of neuronal cells.

In additional embodiments, the provided methods include further administering an additional therapeutic agent to the subject.

### Neurodegenerative Disease

Neurodegenerative diseases are disorders of the central nervous system that are characterized by progressive, normally gradual, loss of functional neural tissue. Neurodegenerative disease, such as Parkinson's disease, Alzheimer's disease, Huntington's disease, affect millions of individuals worldwide. In the United States alone, nearly one million people are currently living with Parkinson's disease. No cure is presently known, although treatment options, including surgery and medications, are available to manage symptoms.

In one embodiment, the disclosure provides a method of treating a neurodegenerative disease in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the neurodegenerative disease treated according to the provided methods is Alzheimer's disease, Parkinson's disease, Huntington's disease, amyotrophic lateral sclerosis (ALS), Friedreich's ataxia, frontotemporal lobar degeneration, Creutzfeldt-Jakob disease, or dementia.

In some embodiments, the neurodegenerative disease treated according to the provided methods is Alzheimer's disease (AD), Parkinson's disease (PD), or Huntington's disease (HD). In a particular embodiment, the treated neurodegenerative disease is AD. In a particular embodiment, the treated neurodegenerative disease is PD. In a particular embodiment, the treated neurodegenerative disease is HD.

In some embodiments, the neurodegenerative disease treated according to the provided methods is selected from: amyotrophic lateral sclerosis (ALS), Friedreich's ataxia, and frontotemporal lobar degeneration. In a particular embodiment, the treated neurodegenerative diseases is ALS.

In some embodiments, the neurodegenerative disease treated according to the provided methods is dementia. In further embodiments, the treated dementia is AD-type senile dementia (SDAT), vascular dementia, dementia with Lewy bodies, or Dementia Pugilistica.

In some embodiments, the subject treated according to the provided methods is at risk of having an neurodegenerative disease. In some embodiments, the provided methods are performed as a prophylactic treatment for a neurodegenerative disease.

In some embodiments, the provided methods prevent a neurodegenerative disease in a subject at risk for developing the neurodegenerative disease, e.g., a subject having one or more risk factors associated with development of the neurodegenerative disease. In some embodiments, the subject has one or more risk factors selected from: over 55 years of age, smoking, obesity, diabetes, heart disease, high blood pressure, stroke, myocardial infarction, family history of a neurodegenerative disease, metabolic syndrome, exposure to solvents, exposure to pesticides, and head injury or brain trauma.

In some embodiments, the subject treated according to the provided methods has, or has been diagnosed as having, a neurodegenerative disease. The development of neurodegenerative diseases can routinely be detected and assessed using standard clinical techniques known in the art, such as functional tests including cognitive tests, behavioral reflex tests; balance, coordination and gait tests, and deep tendon reflex tests); qualitative examinations of mental status, cranial nerves, motor system, sensory system; biochemical assays; nerve and muscle biopsy; and computerized axial tomography (CAT) scan, computerized tomography (CT), nuclear magnetic resonance (MRI), Transcranial Doppler, neurosonography, electroencephalogram (EEG), SPECT scan, positive electricity Sub-emission computed tomography art (PET), or diffusion-weighted imaging (DWI).

In some embodiments, the disclosure provides methods and compositions that prevent, inhibit or delay the onset of an neurodegenerative disease by administering compositions provided herein to a subject before the onset of the neurodegenerative disease, e.g., before the onset of one or more symptoms thereof. In some embodiments, the HIF1-α Pathway Inhibitor and the PFKFB3 inhibitor are administered before the onset of one or more symptoms of the neurodegenerative disease. In some embodiments, treating a neurodegenerative disease according to a method provided herein comprises delaying the onset of one or more symptoms of a neurodegenerative disease.

In some embodiments, the HIF1-α Pathway Inhibitor and the PFKFB3 inhibitor are administered after the onset of one or more symptoms of a neurodegenerative disease. In some embodiments, the HIF1-α Pathway Inhibitor and the PFKFB3 inhibitor are administered to inhibit the progression of a neurodegenerative disease. In some embodiments, the provided methods and compositions are used to treat one or more different stages of a neurodegenerative disease.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating a neurodegenerative disease is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating a neurodegenerative disease according to a method provided herein comprises reducing one or more symptoms of the neurodegenerative disease in the subject compared to the subject prior to treatment with the HIF1-α Pathway Inhibitor and the PFKFB3 inhibitor.

In some embodiments, the one or more reduced symptoms of the neurodegenerative disease is selected from: memory loss, cognitive impairment, personality changes, depression, mood swings, unsteady gait, twitching and jerking movements and tremors, dementia, slurred speech, drooling, impaired judgement, difficulty in swallowing, rigid muscles, impaired posture and balance; loss of automatic movements, difficulty writing, reduction in apoptosis/destruction/loss of the number and/or function of neural cells and/or tissue; increase survival of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; and increased survival/survival time. In some embodiments, the one or more symptoms of neurodegenerative disease are reduced by at least 10%, at least 20%, at least 30%, at least 40%, or at least 50% compared to the subject prior to treatment with the HIF1-α Pathway Inhibitor and the PFKFB3 inhibitor.

Treatment and/or prevention of an neurodegenerative disease can be measured by a variety of means routinely used in the art. In some embodiments, treatment comprises reducing or eliminating one or more of the above symptoms. In some embodiments, treatment comprises reducing or eliminating one or more of apoptosis or inflammation, of neural cells or tissue, or reducing or eliminating cognitive, motor and/or sensory impairment in a subject having a neurodegenerative disease.

Neurological tests for identifying and monitoring a neurodegenerative disease are known in the art. The term “neurological test,” as used herein, refers to any test, presently known or unknown, that is useful for ascertaining and/or measuring neurological activity and encompasses anatomically-based testing, functional testing, and biochemical assays. Exemplary neurological tests include, without limitation, CAT scan, nuclear magnetic resonance (MRI), Transcranial Doppler, neurosonography, electroencephalogram (EEG), SPECT scan, computerized tomography (CT) scanning, positive electricity Sub-emission computed tomography art (PET), diffusion-weighted imaging (DWI), as well as more qualitative examinations of mental status, cranial nerves, motor system, sensory system, the deep tendon reflexes, coordination, and gait.

In some embodiments, treating a neurodegenerative disease according to a method provided herein reduces one or more cognitive, behavioral and/or physical impairments in the subject. In some embodiments, treating a neurodegenerative disease according to a method provided herein comprises increasing or improving one or more neurological parameters in the subject compared to the parameter in the subject prior to treatment with the HIF1-α Pathway Inhibitor and the PFKFB3 inhibitor. In some embodiments, the one or more improved neurological parameters are selected from impaired cognition and/or memory; impaired balance, coordination and/or gait; impaired reflexes; tremors, twitching and/or jerking movements; mood swings; dementia; impaired speech and/or writing; and loss of neuronal cells. These parameters may be monitored through routine techniques described herein or otherwise known in the art.

In some embodiments, the administration of the provided compositions have use in (i) improving cognition and/or (ii) treating and/or preventing cognitive impairment. In some embodiments, the administration of the provided compositions have use in (i) improving cognition and/or (ii) treating and/or preventing cognitive dysfunction in a subject. The cognitive function in human subjects can routinely be determined using methods known in the art including for example, the RAVLT cognitive test, Repeatable Battery for the Assessment of Neuropsychological Status, or California Verbal Learning Test). In some embodiments, the cognition of the subject is assessed using a Wechsler memory scale [WMS]-verbal paired associates (VPA) test; a Rey Auditory Verbal Learning test recall [RAVLT recall], a Recognition test, a WMS digit span test, a Controlled Word Association test [COWAT], a category fluency test, a Trail Making Test [TMT], an orientation task ADAS-cog test, or a letter digit substitution test.

In some embodiments, the provided methods prevent, reduce or delay the progression a neurodegenerative disease. The methods may be administered to patients at risk for developing a neurodegenerative disease. In such subjects, prevention of a neurodegenerative disease may be monitored by the absence of typical hallmarks of the neurodegenerative disease. For example, subjects to whom an effective amount of a HIF1-alpha inhibitor and PFKFB3 inhibitor is administered prophylactically may not experience or may experience a reduced incidence of one or more of the following symptoms: impaired cognition and/or memory; impaired balance, coordination and/or gait; impaired reflexes; tremors, twitching and/or jerking movements; mood swings; dementia; impaired speech and/or writing; and loss of neuronal cells.

In additional embodiments, the provided methods include further administering an additional therapeutic agent to the subject.

### Alzheimer's Disease

Alzheimer's disease (AD) is an age-related neurodegenerative disease that is at present, the most common cause of dementia. Clinically, AD is characterized by a progressive decline in cognitive function that is associated with memory deficit together with dysphasia (language disorder in which there is an impairment of speech any of comprehension of speech), dyspraxia (disability to coordinate and perform certain purposeful movements and gestures in the absence of motor or sensory impairments) and agnosia (ability to recognize objects, persons, sounds, shapes, or smells) attributable to involvement of the cortical association areas of the brain. Exemplary clinical symptoms of AD include, mild forgetfulness, difficulty solving simple math problems; trouble remembering how to do simple tasks; inability to think clearly; difficulty speaking, understanding, reading, or writing; confusion, irritability, mood swings, anxiety, aggressiveness, or tendency to wander away from home.

In some embodiments, the disclosure provides methods and compositions for treating Alzheimer's disease (AD) in a subject comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having AD. In some embodiments, a method provided herein (e.g., any of (a)-(c) above), is performed as a prophylactic treatment for AD.

In some embodiments, the provided methods and compositions prevent AD in a subject at risk for developing AD, e.g., a subject having one or more risk factors associated with development of AD. In some embodiments, the subject has one or more risk factors selected from: over 65 years of age, smoking, alcohol, family history of AD, head injury or brain trauma, diabetes, and heart disease.

In some embodiments, the subject has AD. In some embodiments, the subject has been diagnosed as having AD. AD may be diagnosed by physicians using any technique known in the art including for example, medical history, mental status tests, physical and neurological exams, diagnostic tests and brain imaging to diagnose AD.

In some embodiments, the disclosure provides methods and compositions that prevent, inhibit or delay the onset of AD by administration to a subject before the onset of AD, e.g., before the onset of one or more symptoms thereof. Among the most consistent risk factors for AD are age, sex, ethnicity, and family history of AD.

In some embodiments, the HIF1-α Pathway Inhibitor and the PFKFB3 inhibitor are administered before the onset of one or more symptoms of AD. In some embodiments, the subject exhibits at least one of the following: memory loss that disrupts daily life; challenges in planning or solving problems; difficulty completing familiar tasks; confusion with time or place; trouble understanding visual images and spatial relationships; new problems with words in speaking or writing; misplacing things and losing the ability to retrace steps; decreased or poor judgment; withdrawal from work or social activities; or changes in mood and personality. In some embodiments, the provided methods and compositions may reduce the incidence, severity, or level of memory impairment, confusion, and difficulty speaking and/or problem solving. In some embodiments, the provided methods and compositions improve the subjects' cognitive status as determined using a mental cognitive status test (e.g., the Mini-Mental State Exam (MMSE) or the Mini-Cog test). In some embodiments, treating AD according to a method provided herein comprises delaying the onset of one or more symptoms of AD.

In some embodiments, the HIF1-α Pathway Inhibitor and the PFKFB3 inhibitor are administered after the onset of one or more symptoms of AD. In some embodiments, the provided methods and compositions can be used to treat different stages of AD.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating AD is performed by co-administering the HIF1-α Pathway Inhibitor and the PFKFB3 inhibitor to the subject.

In some embodiments, treating AD according to a method provided herein comprises reducing one or more symptoms of AD in the subject compared to the subject prior to treatment with the HIF1-α Pathway Inhibitor and the PFKFB3 inhibitor. In some embodiments, the one or more reduced symptoms of AD is selected from: memory loss that disrupts daily life; challenges in planning or solving problems; difficulty completing familiar tasks; confusion with time or place; trouble understanding visual images and spatial relationships; new problems with words in speaking or writing; misplacing things and losing the ability to retrace steps; decreased or poor judgment; withdrawal from work or social activities; or changes in mood and personality. In some embodiments, treating AD according to a method provided herein is indicated by improved mental cognitive status, e.g., a determined using a mental cognitive status test (e.g., the Mini-Mental State Exam (MMSE) or the Mini-Cog test). In some embodiments, the improved mental cognitive status is indicated by an improved score on a mental cognitive status test of at least 10%, at least 20%, at least 30%, at least 40%, or at least 50% compared to the subject's cognitive score on the corresponding test prior to treatment with the HIF1-α Pathway Inhibitor and the PFKFB3 inhibitor.

Treatment of AD can be measured by a variety of means using techniques known in the art. In some embodiments, treatment comprises reducing one or more of the following AD symptoms in the subject: forgetfulness, difficulty solving simple math problems; trouble remembering how to do simple tasks; inability to think clearly; difficulty speaking, understanding, reading, or writing; confusion, irritability, mood swings, anxiety, aggressiveness, or tendency to wander away from home.

In some embodiments, treating AD according to a method provided herein comprises increasing one or more parameters selected from: maintaining/improving quality of life, maximizing function in daily activities, enhancing cognition, improving mood and improving behavior of the subject.

In some embodiments, the provided methods prevent AD. The methods may be administered to patients at risk for developing AD. In such subjects, prevention of AD may be monitored by lack of typical hallmarks of AD. For example, subjects to whom an effective amount of a HIF1-alpha inhibitor and PFKFB3 inhibitor is administered prophylactically may not experience or may experience a reduced incidence of one or more of the following symptoms: memory loss that disrupts daily life; challenges in planning or solving problems; difficulty completing familiar tasks; confusion with time or place; trouble understanding visual images and spatial relationships; new problems with words in speaking or writing; misplacing things and losing the ability to retrace steps; decreased or poor judgment; withdrawal from work or social activities; or changes in mood and personality.

In some embodiments, the provided methods delay the onset of AD. Accordingly, the provided methods delay the average onset of AD to greater than 5 years, greater than 10 years, greater than 11 years, greater than 12 years, greater than 13 years, greater than 14 years, greater than 15 years, greater than 16 years, greater than 17 years, greater than 18 years, greater than 19 years, or greater than 20 years after initial diabetes diagnosis.

In some embodiments, the provided methods reduce, ameliorate, lessen the severity of, or reverse one or more symptoms of AD. In some embodiments, methods of treating AD with anti-HIF1-alpha and/or anti-PFKFB3 antibodies or antigen-binding fragments thereof may reduce, ameliorate, lessen the severity of, or reverse one or more of the following symptoms of AD: memory loss that disrupts daily life; challenges in planning or solving problems; difficulty completing familiar tasks; confusion with time or place; trouble understanding visual images and spatial relationships; new problems with words in speaking or writing; misplacing things and losing the ability to retrace steps; decreased or poor judgment; withdrawal from work or social activities; or changes in mood and personality.

In additional embodiments, the provided methods include further administering an additional therapeutic agent to the subject. In one embodiment, the additional administered therapeutic agent is an acetyl cholinesterase inhibitor. In one embodiment, the additional administered therapeutic agent is an NMDA receptor antagonist. In another embodiment, the additional administered therapeutic agent is memantine.

### Parkinson's Disease

Parkinson's disease (PD) is a degenerative disorder of the central nervous system, which results from the death of dopamine-generating (DA) neurons in the substantia nigari, a region of the midbrain. PD affects nearly a million Americans, with 50,000 new cases diagnosed in the U.S. each year. PD is the second most common neurodegenerative disorder after Alzheimer's disease. The prevalence of PD is about 0.3% of the whole population in industrialized countries. While genetically linked familial PD has early onset (between the ages of 20 and 50), the more common sporadic PD is occurring after the age of 50 (average 57±11).

The main motor symptoms of PD are collectively called Parkinsonism, or a “parkinsonian syndrome” Early in the course of PD, the most obvious symptoms are movement-related, such as shaking, rigidity, postural instability, and slowness of movement, difficulty with walking, impaired gait and tendency to fall. Later, cognitive and behavioral problems may arise, with dementia commonly occurring in the advanced stages of the disease.

In some embodiments, the disclosure provides methods and compositions for treating PD in a subject comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having PD. In some embodiments, a method provided herein (e.g., any of (a)-(c) above), is performed as a prophylactic treatment for PD.

In some embodiments, the provided methods and compositions prevent PD in a subject at risk for developing PD, e.g., a subject having one or more risk factors associated with development of PD. In some embodiments, the subject is male over 60, and/or has had ongoing exposure to herbicides or pesticides.

In some embodiments, the disclosure provides methods and compositions that prevent, inhibit or delay the onset of PD by administration to a subject before the onset of PD, e.g., before the onset of one or more symptoms thereof.

In some embodiments, the HIF1-α Pathway Inhibitor and the PFKFB3 inhibitor are administered before the onset of one or more symptoms of PD. In some embodiments, the provided methods prevent PD. The methods may be administered to patients at risk for developing PD. In such subjects, prevention of PD may be monitored by lack of typical hallmarks of PD. For example, subjects to whom an effective amount of a HIF1-alpha inhibitor and PFKFB3 inhibitor is administered prophylactically may not experience or may experience a reduced incidence of one or more of the following symptoms: tremors (or shaking, e.g., in a limb, often a hand or fingers), slowed movement (bradykinesia), rigid muscles, impaired posture and balance; loss of automatic movements, speech changes, or difficulty writing.

In some embodiments, the subject has been diagnosed as having PD. There are presently no single test or scan for Parkinson's, but bradykinesia (slowness of movement) and tremor or rigidity are telltale symptoms that help doctors make a diagnosis.

In some embodiments, the HIF1-α Pathway Inhibitor and the PFKFB3 inhibitor are administered after the onset of one or more symptoms of PD. The progression of PD has 5 stages. In some embodiments, the provided methods and compositions can be used to treat different stages of PD.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is Antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating PD is performed by co-administering the HIF1-α Pathway Inhibitor and the PFKFB3 inhibitor to the subject.

In some embodiments, treating PD according to a method provided herein comprises reducing one or more symptoms of PD in the subject compared to the subject prior to treatment with the HIF1-α Pathway Inhibitor and the PFKFB3 inhibitor. In some embodiments, the one or more reduced symptoms of PD is selected from: tremors (or shaking, e.g., in a limb, often a hand or fingers), slowed movement (bradykinesia), rigid muscles, impaired posture and balance; loss of automatic movements, speech changes, difficulty writing. In some embodiments, treating PD according to a method provided herein is indicated by improved mental cognitive status, e.g., as determined using a mental cognitive status test (e.g., the Mini-Mental State Exam (MMSE) or the Mini-Cog test). In some embodiments, the one or more symptoms of PD. In some embodiments, the improved mental cognitive status is indicated by an improved score on a mental cognitive status test of at least 10%, at least 20%, at least 30%, at least 40%, or at least 50% compared to the subject's cognitive score on the corresponding test prior to treatment with the HIF1-α Pathway Inhibitor and the PFKFB3 inhibitor.

Treatment of PD can be measured by a variety of means using techniques known in the art. In some embodiments, treatment comprises reducing one or more of the following PD symptoms in the subject: tremors (or shaking, e.g., in a limb, often a hand or fingers), slowed movement (bradykinesia), rigid muscles, impaired posture and balance; loss of automatic movements, speech changes, and difficulty writing.

In some embodiments, treating PD according to a method provided herein comprises increasing/improving one or more parameters selected from: reduced muscle rigidity, improved ability to walk, reduced involuntary movements, reduced fatigue, reduced dizziness, improved sleep, maintaining/improving quality of life, maximizing function in daily activities, enhancing cognition, improving mood and improving behavior of the subject.

In additional embodiments, the provided methods include further administering an additional therapeutic agent to the subject. In one embodiment, the additional administered therapeutic agent is a dopamine agonist. In a further embodiment, the additional administered therapeutic agent is levodopa.

### Huntington's Disease

Huntington's disease (HD) is a devastating and progressive genetic neurodegenerative disorder having symptoms that can include chorea, rigidity, writhing motions, physical instability, difficulties chewing, swallowing, and speaking, sleep disturbances, cognitive dysfunction, memory deficits, anxiety, depression, aggression, compulsive behavior. Physical symptoms of Huntington's Disease typically occur between 35 and 44 years of age. Life expectancy is around 20 years from the onset of physical symptoms.

In some embodiments, the disclosure provides methods and compositions for treating Huntington's disease (HD) in a subject comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having HD. In some embodiments, a method provided herein (e.g., any of (a)-(c) above), is performed as a prophylactic treatment for HD.

In some embodiments, the HIF1-α Pathway Inhibitor and the PFKFB3 inhibitor are administered before the onset of one or more symptoms of HD. In some embodiments, the disclosure provides methods and compositions that prevent, inhibit or delay the onset of HD by administration to a subject before the onset of HD, e.g., before the onset of one or more symptoms thereof.

In some embodiments, the provided methods and compositions prevent HD in a subject at risk for developing HD, e.g., a subject having one or more risk factors associated with development of HD. In some embodiments, the subject has a family history of HD, or a mutation in the HTT gene (e.g., a CAG repeat mutation).

In some embodiments, the subject has, or has been diagnosed as having HD. Preliminary HD diagnosis of HD typically occurs though a general physical exam, a review family medical history, and neurological and/or psychiatric examinations.

In some embodiments, the disclosure provides methods and compositions that prevent, inhibit or delay the onset of HD by administering compositions provided herein to a subject before the onset of HD, e.g., before the onset of one or more symptoms thereof. In some embodiments, treating HD according to a method provided herein comprises delaying the onset of one or more symptoms of HD.

In some embodiments, the provided methods prevent HD. The methods may be administered to patients at risk for developing HD. In such subjects, prevention of HD may be monitored by lack of typical hallmarks of HD. For example, subjects to whom an effective amount of a HIF1-alpha inhibitor and PFKFB3 inhibitor is administered prophylactically may not experience or may experience a reduced incidence of one or more of the following symptoms: personality changes, depression, mood swings, unsteady gait, involuntary chorea, twitching and jerking movements and tremors, dementia, slurred speech, impaired judgement, difficulty in swallowing and an intoxicated appearance.

In some embodiments, the HIF1-α Pathway Inhibitor and the PFKFB3 inhibitor are administered after the onset of one or more symptoms of HD.

In some embodiments, the provided methods and compositions can be used to treat different stages of HD. The symptoms of Huntington's disease typically appear between the age of about 30 to 50 years and the disease usually progresses over a 10 to 25 year period. The characteristics and symptoms of the disease include personality changes, depression, mood swings, unsteady gait, involuntary chorea, twitching and jerking movements and tremors, dementia, slurred speech, impaired judgement, difficulty in swallowing and an intoxicated appearance. Once an individual becomes symptomatic for Huntington's disease the course of the disease can be roughly divided into three stages (the early, middle and late stages) that collectively can last anywhere from 10-30 years. In the early stage of HD, patients are able to perform most of their usual activities, involuntary movements are generally mild, speech is typically clear, and dementia, if present, is mild. During the early stage, patients may exhibit slight uncontrollable movements, stumbling and clumsiness, lack of concentration, short-term memory lapses and depression, as well as mood swings.

During the middle stage of HD, patients become more disabled and typically need assistance with some of their routine daily activities. Falls, weight loss, and swallowing difficulties may become a problem during the middle stage of HD, with dementia and uncontrollable movements becoming more pronounced.

During the late stage of HD, patients deteriorate to the point where they require almost total care. At this stage, they may no longer be able to walk or speak, their involuntary movements become more rigid, and they are often unaware of their surroundings and unable to swallow food.

In some embodiments, the provided methods are administered to a subject during the early stage of HD. In some embodiments, the provided methods are administered to a subject during the middle stage of HD In some embodiments, the provided methods are administered to a subject during the late stage of HD.

In some embodiments, the subject treated according to a method provided herein exhibits at least one of the following symptoms of HD: difficulty concentrating, memory lapses, depression, stumbling and clumsiness, or mood swings such as irritability and aggressive behavior. In some embodiments, the provided methods and compositions may reduce the incidence, severity, or level of memory impairment, confusion, and difficulty speaking and/or problem solving. In some embodiments, the provided methods and compositions improve the subject's cognitive status as determined using a mental cognitive status test (e.g., the Mini-Mental State Exam (MMSE) or the Mini-Cog test). In some embodiments, treating HD according to a method provided herein comprises delaying the onset of one or more symptoms of HD.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is an antisense oligonucleotide EZN-2968 or a nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating HD is performed by co-administering the HIF1-α Pathway Inhibitor and the PFKFB3 inhibitor to the subject.

In some embodiments, treating HD according to a method provided herein comprises reducing one or more symptoms of HD in the subject compared to the subject prior to treatment with the HIF1-α Pathway Inhibitor and the PFKFB3 inhibitor. In some embodiments, the one or more reduced symptoms of HD is selected from: personality changes, depression, mood swings, unsteady gait, involuntary chorea, twitching and jerking movements and tremors, dementia, slurred speech, impaired judgement, difficulty in swallowing and an intoxicated appearance. In some embodiments, treating HD according to a method provided herein is indicated by improved mental cognitive status, e.g., as determined using a mental cognitive status test (e.g., the Mini-Mental State Exam (MMSE) or the Mini-Cog test). In some embodiments, the one or more symptoms of HD. In some embodiments, the improved mental cognitive status is indicated by an improved score on a mental cognitive status test of at least 10%, at least 20%, at least 30%, at least 40%, or at least 50% compared to the subject's cognitive score on the corresponding test prior to treatment with the HIF1-α Pathway Inhibitor and the PFKFB3 inhibitor.

Treatment of HD can be measured by a variety of means using techniques known in the art. In some embodiments, treatment comprises reducing one or more of the following HD symptoms in the subject: personality changes, depression, mood swings, unsteady gait, involuntary chorea, twitching and jerking movements and tremors, dementia, slurred speech, impaired judgement, difficulty in swallowing and an intoxicated appearance.

In some embodiments, treating HD according to a method provided herein comprises increasing one or more parameters selected from: maintaining/improving quality of life, maximizing function in daily activities, enhancing cognition, improving mood and improving behavior of the subject.

In additional embodiments, the provided methods include further administering an additional therapeutic agent to the subject. In one embodiment, the additional administered therapeutic agent is Tetrabenazine.

### Neurotrauma

In some embodiments, the disclosure provides methods and compositions for treating neurotrauma (e.g., traumatic brain injury, stroke, brain or spinal cord hemorrhage, brain infarct, and spinal cord injury). In some embodiments, the disclosure provides methods and compositions for treating neurotrauma (NT) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having NT. In some embodiments, a method provided herein (e.g., any of (a)-(c) above), is performed as a prophylactic treatment for NT. In some embodiments, the provided methods and compositions prevent NT in a subject at risk for developing NT. Subjects at risk of trauma include those about to undergo surgery, those involved in combat (e.g. military combat), and contact sports (e.g., rugby, football, boxing and mixed martial arts), and accidents (e.g., automobile accidents).

In some embodiments, the subject has NT or has been diagnosed as having NT. NT may be diagnosed by physicians using any technique known in the art including for example, through biomarker testing, clinical response testing, computed tomography (CT), and magnetic resonance imaging (MRI).

A patient may be suspected of having a NT injury on the basis of neurologic symptoms (motor, sensory, cognitive) and/or radiological evaluation (MRI, CT scan, X-ray) consistent with NT, e.g., after a physician's exam. In some embodiments, a patient suspected of having NT, particularly a spinal cord injury, may having a rating of A or B on the American Spinal Cord Injury Association (ASIA) Impairment Scale. The ASIA Impairment Scale is a standard diagnostic tool that assess a patient's motor and sensory function.

In some embodiments the diagnosis of neurotrauma includes the determination of an increased biomarker associated with neurotrauma in a biological sample from the subject. In some embodiments, the increased biomarker in the biological sample of the subject is at least one selected from: aldolase C (ALDOC), brain lipid binding protein (BLBP/FABP7), a trauma-specific break down product (BDP) of ALDOC or BLBP/FABP7, apolipoprotein B (APOB), prostaglandin synthase (PTGDS), glutamine synthetase (GS), astrocytic phosphoprotein PEA-15 (PEA15), aB-crystallin (CRYAB/HSP27), ubiquitin C-terminal hydrolase (UCH-L1), glial fibrillary acidic protein (GFAP), pNF-H, LPA, an LPA metabolite, SBDP150, SBDP150i, SBDP145, SBDP120, MAP2, NLRP1 (NALP-1), ASC, caspase-1, and 12-HETE.

As used herein, “biological sample” refers to any bodily fluid or tissue obtained from a patient or subject. A biological sample can include, but is not limited to, whole blood, red blood cells, plasma, serum, peripheral blood mononuclear cells (PBMCs), urine, saliva, tears, buccal swabs, cerebrospinal fluid (CSF), CNS microdialysate, and nerve tissue. In some embodiments, the biological sample is CSF, saliva, serum, plasma, or urine. In certain embodiments, the biological sample is CSF.

In some embodiments, the disclosure provides methods and compositions that prevent, inhibit or delay the onset of NT by administration to a subject before the onset of NT, e.g., before the onset of one or more symptoms thereof.

In some embodiments, the provided methods prevent NT. The methods may be administered to patients at risk for developing NT. In such subjects, prevention of NT may be monitored by lack of typical hallmarks of NT. For example, subjects to whom an effective amount of a HIF1-alpha inhibitor and PFKFB3 inhibitor is administered prophylactically may not experience or may experience a reduced incidence of one or more of the following symptoms: impaired physical abilities, impaired movement abilities of extremities, impaired ability to coordinate muscle groups, impaired sensory function, impaired cognitive abilities, headaches and/or dizziness.

In some embodiments, the HIF1-α Pathway Inhibitor and the PFKFB3 inhibitor are administered after the onset of one or more symptoms of NT. In some embodiments, the provided methods: reduce the damage to nerve cells or nerve tissue; reduce inflammation of nerve cells or nerve tissue; reduce neurodegeneration or death of nerve cells or nerve tissue; improve functionality of nerve cells or nerve tissue; improve functional recovery of nerve cells or nerve tissue, such as locomotor improvement. In some embodiments, the provided methods improve the subjects' cognitive status as determined using a mental cognitive status test (e.g., the Mini-Mental State Exam (MMSE) or the Mini-Cog test). In some embodiments, treating NT according to a method provided herein comprises delaying the onset of one or more symptoms of NT.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In some embodiments, the administered PFKFB3 inhibitor is AZ67, or a salt thereof.

In some embodiments, the administered PFKFB3 inhibitor has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In some embodiments, the administered PFKFB3 inhibitor has the structure of formula AZ44-AZ70 or AZ71, depicted in FIG. 1E, or a salt thereof.

In some embodiments, a method provided herein for treating NT is performed by co-administering the HIF1-α Pathway Inhibitor and the PFKFB3 inhibitor to the subject.

In some embodiments, treating NT according to a method provided herein comprises reducing one or more symptoms of NT in the subject compared to the subject prior to treatment with the HIF1-α Pathway Inhibitor and the PFKFB3 inhibitor. In some embodiments, the one or more reduced symptoms of NT is selected from: impaired physical abilities, impaired movement abilities of extremities, impaired ability to coordinate muscle groups, impaired sensory function, impaired cognitive abilities, headaches and/or dizziness. In some embodiments, treating NT according to a method provided herein is indicated by improved mental cognitive status, e.g., a determined using a mental cognitive status test (e.g., the Mini-Mental State Exam (MMSE) or the Mini-Cog test). In some embodiments, the improved mental cognitive status is indicated by an improved score on a mental cognitive status test of at least 10%, at least 20%, at least 30%, at least 40%, or at least 50% compared to the subject's cognitive score on the corresponding test prior to treatment with the HIF1-α Pathway Inhibitor and the PFKFB3 inhibitor.

In some embodiments, treating a NT according to a method provided herein comprises reducing one or more symptoms of the NT in the subject compared to a control or then compared to the subject prior to treatment with the HIF1-α Pathway Inhibitor and the PFKFB3 inhibitor. In some embodiments, the one or more reduced symptoms of the NT is selected from: reduction in apoptosis/destruction/loss of the number and/or function of neural cells and/or tissue; increase in survival and/or function of neural cells and/or tissue; reduction in long-term damage to neural cells/tissue and/or to surrounding cells/tissue; decrease of the inflammation in neural cells/tissues; reduction in the oxidative stress in neural cells/tissues; and increased survival/survival time. In some embodiments, the one or more symptoms of the neural trauma are reduced by at least 10%, at least 20%, at least 30%, at least 40%, or at least 50% compared to a control subject or compared to the subject prior to treatment with the HIF1-α Pathway Inhibitor and the PFKFB3 inhibitor.

In some embodiments, the provided methods result in decreased apoptosis and/or cell death of neural cells or tissue in the subject. Cell death can be monitored according to known methods. Illustrative methods for detecting cell death include but are not limited to, nuclear staining techniques such as propidium iodide, Hoechst-33342, 4′, 6-diamidino-2-phenylindole (DAPI), and Acridine orange-Ethidium bromide staining. Nonnuclear staining techniques include but are not limited to, Annexin-V staining.

Also provided are methods for reducing the size of a brain infarct in subjects known or suspected to have sustained neurotrauma, as well methods for increasing locomotor recovery in such subjects.

In some embodiments, the disclosure provides a method of treating a subject suffering from, or at risk of, a condition characterized by neuronal death and suboptimal neuronal survival Such conditions include neurotrauma, traumatic brain injury, spinal cord injury, or other mechanistic events causing injury to the brain, like surgical measures, e.g. those occurring after brain tumor surgery

Treatment of NT can be measured by a variety of means using techniques known in the art. In some embodiments, treatment comprises reducing one or more of the following NT symptoms in the subject impaired physical abilities, impaired movement abilities of extremities, impaired ability to coordinate muscle groups, impaired sensory function, impaired cognitive abilities, headaches and/or dizziness.

In additional embodiments, the provided methods include further administering an additional therapeutic agent to the subject.

The disclosure of each of U.S. Appl. No. 63/189,204, U.S. Appl. No. 63/189,205, U.S. Appl. No. 63/189,206, and U.S. Appl. No. 63/189,207, each filed May 16, 2021, is herein incorporated by reference in its entirety.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating a neurological condition in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

2. The method of claim 1, wherein the subject is administered an effective amount of the HIF1-α Pathway Inhibitor and the PFKFB3 inhibitor.

3. The method of claim 1, wherein the subject is administered an effective amount of the HIF1-α Pathway Inhibitor and wherein the subject has previously been administered the PFKFB3 Inhibitor.

4. The method of claim 1, wherein the subject is administered an effective amount of the PFKFB3 Inhibitor and wherein the subject has previously been administered the HIF1-α Pathway Inhibitor.

5. The method of any one of claims 1-4, wherein the method of any one of claim 1(a)-1(c) is administered as a prophylactic treatment for the neurological condition.

6. The method of any one of claims 1-4, wherein the subject has or is at risk of having the neurological condition.

7. The method of any one of claims 1-4, wherein the subject has or has been diagnosed as having the neurological condition.

8. The method of any one of claims 1-7, wherein the neurological condition is a neurodegenerative disease, dementia, neuropathy, or neurotrauma.

9. The method of any one of claims 1-8, wherein the neurological condition is a neurodegenerative disease.

10. The method of any one of claims 1-8, wherein the neurological condition is a dementia.

11. The method of any one of claims 1-8, wherein the neurological condition is a neuropathy.

12. The method of any one of claims 1-8, wherein the neurological condition is neurotrauma such as a spinal cord injury or a traumatic brain injury.

13. The method of any one of claims 1-12 wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor.

14. The method of any one of claims 1-13 wherein the administered HIF1-α Pathway Inhibitor is silibinin, PX-478 or YC-1, or a salt thereof.

15. The method of any one of claims 1-14 wherein the administered HIF1-α Pathway Inhibitor is ganetespib (ST-9090), phenethyl isothiocyanate, or BAY-87-2243, or a salt thereof.

16. The method of any one of claims 1-15 wherein the administered HIF1-α Pathway Inhibitor is a HIF1-α Inhibitor.

17. The method of claim 16 wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

18. The method of claim 16 or 17, wherein the administered HIF1-α Inhibitor is antisense oligonucleotide EZN-2968 or nanobody AG-1, AG-2, AG-3, AG-4, AG-5, VHH212, or AHPC.

19. The method of any one of claims 1-18, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

20. The method of any one of claims 1-19, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

21. The method of any one of claims 1-20, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

22. The method of any one of claims 1-21, wherein the HIF1-α Pathway Inhibitor and the PFKFB3 inhibitor are co-administered to the subject.

23. The method of any one of claims 1-22, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

24. The method of any one of claims 1-23, wherein the HIF1-α Pathway Inhibitor and the PFKFB3 inhibitor are administered before the onset of one or more symptoms of the neurological condition.

25. The method of any one of claims 1-24, wherein treating the neurological condition comprises delaying the onset of the neurological condition.

26. The method of any one of claims 1-23, wherein the HIF1-α Pathway Inhibitor and the PFKFB3 inhibitor are administered after the onset of one or more symptoms of the neurological condition.

27. The method of claim 26, wherein the method results in one or more symptoms of the neurological condition are reduced in the subject administered the HIF1-α Pathway Inhibitor and the PFKFB3 inhibitor compared to in the subject prior to treatment.

28. The method of claim 27, wherein the one or more reduced symptoms of the neurological condition is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time.

29. The method of claim 27 or 28, wherein the one or more symptoms of the neurological condition are reduced by at least 10%, at least 20%, at least 30%, at least 40%, or at least 50% compared to in the subject prior to treatment with the HIF1-α Pathway Inhibitor and the PFKFB3 inhibitor.

30. The method according to any one of claims 27-29, wherein at least one of the behavioral reflexes; cognitive skills, balance, coordination, and cognitive skills of the subject are improved compared to in the subject prior to treatment with the HIF1-α Pathway Inhibitor and the PFKFB3 inhibitor.

31. The method according to any one of claims 27-30, wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor.

32. The method of any one of claims 1-31, which further comprises administering an additional Therapeutic agent to the subject.

33. A method of treating a neurodegenerative disease in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

34. The method of claim 33, wherein the subject is administered an effective amount of the HIF1-α Pathway Inhibitor and the PFKFB3 inhibitor.

35. The method of claim 33, wherein the subject is administered an effective amount of the HIF1-α Pathway Inhibitor and wherein the subject has previously been administered the PFKFB3 Inhibitor.

36. The method of claim 33, wherein the subject is administered an effective amount of the PFKFB3 Inhibitor and wherein the subject has previously been administered the HIF1-α Pathway Inhibitor.

37. The method of any one of claims 33-36, wherein the method of any one of claim 33(a)-33(c) is administered as a prophylactic treatment for the neurodegenerative disease.

38. The method of any one of claims 33-36, wherein the subject has or is at risk of having the neurodegenerative disease.

39. The method of any one of claims 33-36, wherein the subject has or has been diagnosed as having the neurodegenerative disease.

40. The method of any one of claims 33-39, wherein the neurodegenerative disease is selected from Alzheimer's disease (AD), Parkinson's disease (PD), Huntington's disease (HD), amyotrophic lateral sclerosis (ALS), Friedreich's ataxia, frontotemporal lobar degeneration, or dementia with Lewy bodies.

41. The method of claim 40, wherein the neurodegenerative disease is Alzheimer's disease (AD).

42. The method of claim 40, wherein the neurodegenerative disease is a Parkinson's disease (PD).

43. The method of claim 40, wherein the neurodegenerative disease is a Huntington's disease (HD.

44. The method of claim 40, wherein the neurodegenerative disease is a amyotrophic lateral sclerosis (ALS).

45. The method of any one of claims 33-44, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

46. The method of any one of claims 33-45, wherein the administered HIF1-α Pathway Inhibitor is silibinin, PX-478 or YC-1, or a salt thereof.

47. The method of any one of claims 33-45, wherein the administered HIF1-α Pathway Inhibitor is ganetespib (ST-9090), phenethyl isothiocyanate, or BAY-87-2243, or a salt thereof.

48. The method of any one of claims 33-47, wherein the administered HIF1-α Pathway Inhibitor is a HIF1-α Inhibitor.

49. The method of claim 48, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

50. The method of claim 48 or 49, wherein the administered HIF1-α Inhibitor is Antisense oligonucleotide EZN-2968 or nanobody AG-1, AG-2, AG-3, AG-4, AG-5, VHH212, or AHPC.

51. The method of any one of claims 33-50, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

52. The method of any one of claims 33-51, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

53. The method of any one of claims 33-51, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

54. The method of any one of claims 33-53, wherein the HIF1-α Pathway Inhibitor and the PFKFB3 inhibitor are co-administered to the subject.

55. The method of any one of claims 33-54, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

56. The method of any one of claims 33-55, wherein the HIF1-α Pathway Inhibitor and the PFKFB3 inhibitor are administered before the onset of one or more symptoms of the neurodegenerative disease.

57. The method of any one of claims 33-56, wherein treating the neurodegenerative disease comprises delaying the onset of the neurodegenerative disease.

58. The method of any one of claims 33-57, wherein the HIF1-α Pathway Inhibitor and the PFKFB3 inhibitor are administered after the onset of one or more symptoms of the neurodegenerative disease.

59. The method of any one of claims 33-58, wherein the method results in reduction in one or more symptoms of the neurodegenerative disease the subject administered the HIF1-α Pathway Inhibitor and the PFKFB3 inhibitor compared to in the subject prior to treatment.

60. The method of claim 59, wherein the one or more reduced symptoms of the neurodegenerative disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells; reduction in the oxidative stress in neural cells/tissues; improvement in behavioral reflexes; improvement of cognitive skills, improved balance and/or coordination and increased survival/survival time.

61. The method of claim 59 or 60, wherein the one or more symptoms of the neurodegenerative disease are reduced by at least 10%, at least 20%, at least 30%, at least 40%, or at least 50% compared to in the subject prior to treatment with the HIF1-α Pathway Inhibitor and the PFKFB3 inhibitor.

62. The method according to any one of claims 59-61 wherein at least one of the behavioral reflexes; balance, coordination, and cognitive skills of the subject are improved compared to in the subject prior to treatment with the HIF1-α Pathway Inhibitor and the PFKFB3 inhibitor.

63. The method according to any one of claims 59-62 wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor.

64. The method of any one of claims 33-63, which further comprises administering an additional Therapeutic agent to the subject.

65. A method of treating Alzheimer's disease (AD) in a subject in need thereof comprising:
(d) administering an effective amount of a HIF1-α Pathway Inhibitor and an PFKFB3 inhibitor to the subject;
(e) administering an effective amount of a HIF1-α Pathway Inhibitor to the subject, wherein the subject has previously been administered a PFKFB3 Inhibitor; or
(f) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

66. The method of claim 65, wherein the subject is administered an effective amount of the HIF1-α Pathway Inhibitor and the PFKFB3 inhibitor.

67. The method of claim 65, wherein the subject is administered an effective amount of the HIF1-α Pathway Inhibitor and wherein the subject has previously been administered the PFKFB3 Inhibitor.

68. The method of claim 65, wherein the subject is administered an effective amount of the PFKFB3 Inhibitor and wherein the subject has previously been administered the HIF1-α Pathway Inhibitor.

69. The method of any one of claims 65-68, wherein the method of any one of claim 65(a)-65(c) is administered as a prophylactic treatment for Alzheimer's disease.

70. The method of any one of claims 65-68, wherein the subject has or is at risk of having Alzheimer's disease.

71. The method of any one of claims 65-68, wherein the subject has or has been diagnosed as having Alzheimer's disease.

72. The method of any one of claims 65-71, wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

73. The method of any one of claims 65-72, wherein the administered HIF1-α Pathway Inhibitor is silibinin, PX-478 or YC-1, or a salt thereof.

74. The method of any one of claims 65-72, wherein the administered HIF1-α Pathway Inhibitor is ganetespib (ST-9090), phenethyl isothiocyanate, or BAY-87-2243, or a salt thereof.

75. The method of any one of claims 65-72, wherein the administered HIF1-α Pathway Inhibitor is a HIF1-α Inhibitor.

76. The method of claim 75, wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

77. The method of claim 75 or 76, wherein the administered HIF1-α Inhibitor is Antisense oligonucleotide EZN-2968 or nanobody AG-1, AG-2, AG-3, AG-4, AG-5, VHH212, or AHPC.

78. The method of any one of claims 65-77, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

79. The method of any one of claims 65-78, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

80. The method of any one of claims 65-78, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

81. The method of any one of claims 65-80, wherein the HIF1-α Pathway Inhibitor and the PFKFB3 inhibitor are co-administered to the subject.

82. The method of any one of claims 65-81, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

83. The method of any one of claims 65-82, wherein the HIF1-α Pathway Inhibitor and the PFKFB3 inhibitor are administered before the onset of one or more symptoms of Alzheimer's disease.

84. The method of any one of claims 65-83, wherein treating Alzheimer's disease comprises delaying the onset of Alzheimer's disease.

85. The method of any one of claims 65-84, wherein the HIF1-α Pathway Inhibitor and the PFKFB3 inhibitor are administered after the onset of one or more symptoms of Alzheimer's disease.

86. The method of any one of claims 65-85, wherein the method results in reduction in one or more symptoms of Alzheimer's disease the subject administered the HIF1-α Pathway Inhibitor and the PFKFB3 inhibitor compared to in the subject prior to treatment.

87. The method of claim 86, wherein the one or more reduced symptoms of Alzheimer's disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time.

88. The method of claim 86 or 87, wherein the one or more symptoms of Alzheimer's disease are reduced by at least 10%, at least 20%, at least 30%, at least 40%, or at least 50% compared to in the subject prior to treatment with the HIF1-α Pathway Inhibitor and the PFKFB3 inhibitor.

89. The method according to any one of claims 86-88, wherein at least one of the behavioral reflexes; balance, coordination, and cognitive skills of the subject are improved compared to in the subject prior to treatment with the HIF1-α Pathway Inhibitor and the PFKFB3 inhibitor.

90. The method according to any one of claims 86-89, wherein at least one of the cognitive skills of the subject are improved by at least 10%, at least 20%, at least 30%, at least 40%, or at least 50%, or at least one of the following symptoms are reduced in the subject compared to in the subject prior to treatment with the HIF1-α Pathway Inhibitor and the PFKFB3 inhibitor: forgetfulness, difficulty solving simple math problems; trouble remembering how to do simple tasks; inability to think clearly; difficulty speaking, understanding, reading, or writing; confusion, irritability, mood swings, anxiety, aggressiveness, or tendency to wander away from home.

91. The method of any one of claims 65-90, which further comprises administering an additional Therapeutic agent to the subject.

92. A method of treating Parkinson's disease (PD) in a subject in need thereof comprising: administering an effective amount of a HIF1-α Pathway Inhibitor and an PFKFB3 inhibitor to the subject:
(a) administering an effective amount of a HIF1-α Pathway Inhibitor to the subject, wherein the subject has previously been administered a PFKFB3 Inhibitor; or
(b) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

93. The method of claim 92, wherein the subject is administered an effective amount of the HIF1-α Pathway Inhibitor and the PFKFB3 inhibitor.

94. The method of claim 92, wherein the subject is administered an effective amount of the HIF1-α Pathway Inhibitor and wherein the subject has previously been administered the PFKFB3 Inhibitor.

95. The method of claim 92, wherein the subject is administered an effective amount of the PFKFB3 Inhibitor and wherein the subject has previously been administered the HIF1-α Pathway Inhibitor.

96. The method of any one of claims 92-95, wherein the method of any one of claim 92(a)-92(c) is administered as a prophylactic treatment for Parkinson's disease.

97. The method of any one of claims 92-95, wherein the subject has or is at risk of having Parkinson's disease.

98. The method of any one of claims 92-95, wherein the subject has or has been diagnosed as having Parkinson's disease.

99. The method of any one of claims 92-98, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

100. The method of any one of claims 92-99, wherein the administered HIF1-α Pathway Inhibitor is silibinin, PX-478 or YC-1, or a salt thereof.

101. The method of any one of claims 92-99, wherein the administered HIF1-α Pathway Inhibitor is ganetespib (ST-9090), phenethyl isothiocyanate, or BAY-87-2243, or a salt thereof.

102. The method of any one of claims 92-99, wherein the administered HIF1-α Pathway Inhibitor is a HIF1-α Inhibitor.

103. The method of claim 102, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

104. The method of claim 102 or 103, wherein the administered HIF1-α Inhibitor is Antisense oligonucleotide EZN-2968 or nanobody AG-1, AG-2, AG-3, AG-4, AG-5, VHH212, or AHPC.

105. The method of any one of claims 92-104, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

106. The method of any one of claims 92-105, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

107. The method of any one of claims 92-105, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

108. The method of any one of claims 92-107, wherein the HIF1-α Pathway Inhibitor and the PFKFB3 inhibitor are co-administered to the subject.

109. The method of any one of claims 92-108, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

110. The method of any one of claims 92-109, wherein the HIF1-α Pathway Inhibitor and the PFKFB3 inhibitor are administered before the onset of one or more symptoms of Parkinson's disease.

111. The method of any one of claims 92-110, wherein treating Parkinson's disease comprises delaying the onset of Parkinson's disease.

112. The method of any one of claims 92-111, wherein the HIF1-α Pathway Inhibitor and the PFKFB3 inhibitor are administered after the onset of one or more symptoms of Parkinson's disease.

113. The method of any one of claims 92-112, wherein the method results in reduction in one or more symptoms of Parkinson's disease the subject administered the HIF1-α Pathway Inhibitor and the PFKFB3 inhibitor compared to in the subject prior to treatment.

114. The method of claim 113, wherein the one or more reduced symptoms of Parkinson's disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time.

115. The method of claim 113 or 114, wherein the one or more symptoms of Parkinson's disease are reduced by at least 10%, at least 20%, at least 30%, at least 40%, or at least 50% compared to in the subject prior to treatment with the HIF1-α Pathway Inhibitor and the PFKFB3 inhibitor.

116. The method according to any one of claims 113-115, wherein at least one of the following symptoms is improved in the subject compared to prior to treatment with the HIF1-α Pathway Inhibitor and the PFKFB3 inhibitor: tremors slowed movement (bradykinesia), rigid muscles, impaired posture and balance; loss of automatic movements, changes in speech, or difficulty writing.

117. The method according to claim 113-116 wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor.

118. The method of any one of claims 92-117, which further comprises administering an additional Therapeutic agent to the subject.

119. A method of treating Huntington's disease (HD) in a subject in need thereof comprising:
(c) administering an effective amount of a HIF1-α Pathway Inhibitor and an PFKFB3 inhibitor to the subject;
(d) administering an effective amount of a HIF1-α Pathway Inhibitor to the subject, wherein the subject has previously been administered a PFKFB3 Inhibitor; or
(e) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

120. The method of claim 119, wherein the subject is administered an effective amount of the HIF1-α Pathway Inhibitor and the PFKFB3 inhibitor.

121. The method of claim 119, wherein the subject is administered an effective amount of the HIF1-α Pathway Inhibitor and wherein the subject has previously been administered the PFKFB3 Inhibitor.

122. The method of claim 119, wherein the subject is administered an effective amount of the PFKFB3 Inhibitor and wherein the subject has previously been administered the HIF1-α Pathway Inhibitor.

123. The method of any one of claims 119-122, wherein the method of any one of claim 119(a)-119(c) is administered as a prophylactic treatment for Huntington's disease.

124. The method of any one of claims 119-122, wherein the subject has or is at risk of having Huntington's disease.

125. The method of any one of claims 119-122, wherein the subject has or has been diagnosed as having Huntington's disease.

126. The method of any one of claims 119-125, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

127. The method of any one of claims 119-126, wherein the administered HIF1-α Pathway Inhibitor is silibinin, PX-478 or YC-1, or a salt thereof.

128. The method of any one of claims 119-126, wherein the administered HIF1-α Pathway Inhibitor is ganetespib (ST-9090), phenethyl isothiocyanate, or BAY-87-2243, or a salt thereof.

129. The method of any one of claims 119-126, wherein the administered HIF1-α Pathway Inhibitor is a HIF1-α Inhibitor.

130. The method of claim 129, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

131. The method of claim 129 or 130, wherein the administered HIF1-α Inhibitor is Antisense oligonucleotide EZN-2968 or nanobody AG-1, AG-2, AG-3, AG-4, AG-5, VHH212, or AHPC.

132. The method of any one of claims 119-131, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

133. The method of any one of claims 119-132, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

134. The method of any one of claims 119-132, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

135. The method of any one of claims 119-134, wherein the HIF1-α Pathway Inhibitor and the PFKFB3 inhibitor are co-administered to the subject.

136. The method of any one of claims 119-135, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

137. The method of any one of claims 119-136, wherein the HIF1-α Pathway Inhibitor and the PFKFB3 inhibitor are administered before the onset of one or more symptoms of Huntington's disease.

138. The method of any one of claims 119-137, wherein treating Huntington's disease comprises delaying the onset of Huntington's disease.

139. The method of any one of claims 119-138, wherein the HIF1-α Pathway Inhibitor and the PFKFB3 inhibitor are administered after the onset of one or more symptoms of Huntington's disease.

140. The method of any one of claims 119-139, wherein the method results in reduction in one or more symptoms of Huntington's disease the subject administered the HIF1-α Pathway Inhibitor and the PFKFB3 inhibitor compared to in the subject prior to treatment.

141. The method of claim 140, wherein the one or more reduced symptoms of Huntington's disease is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time.

142. The method of claim 140 or 141, wherein the one or more symptoms of Huntington's disease are reduced by at least 10%, at least 20%, at least 30%, at least 40%, or at least 50% compared to in the subject prior to treatment with the HIF1-α Pathway Inhibitor and the PFKFB3 inhibitor.

143. The method according to any one of claims 140-142, %, or at least one of the following symptoms are reduced in the subject compared to in the subject prior to treatment with the HIF1-α Pathway Inhibitor and the PFKFB3 inhibitor: memory impairment, confusion, impaired judgement slurred speech, difficulty problem solving, personality changes, depression, mood swings, unsteady gait, involuntary chorea, twitching and jerking movements and tremors, and dementia.

144. The method according to any one of claims 140-143 wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor.

145. The method of any one of claims 119-144, which further comprises administering an additional Therapeutic agent to the subject.

146. A method of treating neurotrauma in a subject in need thereof comprising:
(f) administering an effective amount of a HIF1-α Pathway Inhibitor and an PFKFB3 inhibitor to the subject;
(g) administering an effective amount of a HIF1-α Pathway Inhibitor to the subject, wherein the subject has previously been administered a PFKFB3 Inhibitor; or
(h) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

147. The method of claim 146, wherein the subject is administered an effective amount of the HIF1-α Pathway Inhibitor and the PFKFB3 inhibitor.

148. The method of claim 146, wherein the subject is administered an effective amount of the HIF1-α Pathway Inhibitor and wherein the subject has previously been administered the PFKFB3 Inhibitor.

149. The method of claim 146, wherein the subject is administered an effective amount of the PFKFB3 Inhibitor and wherein the subject has previously been administered the HIF1-α Pathway Inhibitor.

150. The method of any one of claims 146-149, wherein the method of any one of claim 146(a)-146(c) is administered as a prophylactic treatment for neurotrauma.

151. The method of any one of claims 146-149, wherein the subject has or is at risk of having neurotrauma.

152. The method of any one of claims 146-149, wherein the subject has or has been diagnosed as having neurotrauma.

153. The method of any one of claims 146-153, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

154. The method of any one of claims 146-153, wherein the administered HIF1-α Pathway Inhibitor is silibinin, PX-478 or YC-1, or a salt thereof.

155. The method of any one of claims 146-153, wherein the administered HIF1-α Pathway Inhibitor is ganetespib (ST-9090), phenethyl isothiocyanate, or BAY-87-2243, or a salt thereof.

156. The method of any one of claims 146-153, wherein the administered HIF1-α Pathway Inhibitor is a HIF1-α Inhibitor.

157. The method of claim 156, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

158. The method of claim 156 or 157, wherein the administered HIF1-α Inhibitor is Antisense oligonucleotide EZN-2968 or nanobody AG-1, AG-2, AG-3, AG-4, AG-5, VHH212, or AHPC.

159. The method of any one of claims 146-158, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

160. The method of any one of claims 146-159, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

161. The method of any one of claims 146-159, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

162. The method of any one of claims 146-161, wherein the HIF1-α Pathway Inhibitor and the PFKFB3 inhibitor are co-administered to the subject.

163. The method of any one of claims 146-162, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

164. The method of any one of claims 146-163, wherein the neurotrauma is spinal cord injury.

165. The method of any one of claims 146-163, wherein the neurotrauma is traumatic brain injury.

166. The method of any one of claims 146-165, wherein the HIF1-α Pathway Inhibitor and the PFKFB3 inhibitor are administered before the onset of one or more symptoms of neurotrauma.

167. The method of any one of claims 146-165, wherein the HIF1-α Pathway Inhibitor and the PFKFB3 inhibitor are administered before the onset of one or more symptoms of neurotrauma.

168. The method of any one of claims 146-167, wherein treating neurotrauma comprises delaying the onset of neurotrauma.

169. The method of any one of claims 146-168, wherein the HIF1-α Pathway Inhibitor and the PFKFB3 inhibitor are administered after the onset of one or more symptoms of neurotrauma.

170. The method of any one of claims 146-169, wherein the method results in reduction in one or more symptoms of neurotrauma the subject administered the HIF1-α Pathway Inhibitor and the PFKFB3 inhibitor compared to in the subject prior to treatment.

171. The method of claim 170, wherein the one or more reduced symptoms of neurotrauma is indicated by: reduction in apoptosis/destruction/loss of the number and/or function of neural cells and/or tissue; increased survival and/or function of neural cells and/or tissue (e.g. neurons); reduction or delay of neurodegeneration, recovery of motor function; reduction in long-term damage to neural cells/tissue and/or to surrounding cells/tissue; decrease of the inflammation in neural cells/tissues; reduction in the oxidative stress in neural cells/tissues; improvement in behavioral reflexes; improvement of cognitive skills, improved balance and/or coordination and increased survival/survival time.

172. The method of claim 170 or 171, wherein the one or more symptoms of neurotrauma are reduced by at least 10%, at least 20%, at least 30%, at least 40%, or at least 50% compared to in the subject prior to treatment with the HIF1-α Pathway Inhibitor and the PFKFB3 inhibitor.

173. The method according to any one of claims 170-172, wherein at least one of the behavioral reflexes; balance, coordination, and cognitive skills of the subject are improved compared to in the subject prior to treatment with the HIF1-α Pathway Inhibitor and the PFKFB3 inhibitor.

174. The method according to any one of claims 170-173 wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor.

175. The method of any one of claims 146-174, which further comprises administering an additional Therapeutic agent to the subject.
